(19) United States
(12) Patent Application Publication
(10) Pub. No.: US 2023/0053523 A1
(43) Pub. Date: Feb. 23, 2023

(54) METHODS AND SYSTEMS FOR IDENTIFYING RECOMBINANT VARIANTS

(71) Applicant: Illumina, Inc., San Diego, CA (US)

(72) Inventors: Xiao Chen, San Diego, CA (US); Michael A. Eberle, San Diego, CA (US)

(21) Appl. No.: 17/833,394

(22) Filed: Jun. 6, 2022

Related U.S. Application Data
(60) Provisional application No. 63/197,936, filed on Jun. 7, 2021.

(51) Int. Cl. G16B 20/10; C12Q 1/6858; C12Q 1/6869; G16B 30/10

(52) U.S. Cl. CPC G16B 20/10; C12Q 1/6858; C12Q 1/6869; G16B 30/10

(57) ABSTRACT

Disclosed herein include systems, devices, and methods for identifying recombinant variants (e.g., gene conversion variants) of genes such as GBA gene and CYP21A2 gene, the copy numbers of recombinant variants, and gene variant status (e.g., carrier, compound heterozygous, or homozygous).

### RELATED APPLICATIONS

The present application claims priority under 35 U.S.C. § 119(e) to U.S. Provisional Application No. 63/197,936, filed Jun. 7, 2021. The content of the related application is incorporated herein by reference in its entirety.

### BACKGROUND

### Field

This disclosure relates generally to the field of determining gene variants, and more particularly to determining recombinant variants.

### Background

Segmental duplications are hotspots for structural variants and gene recombinant variants (for example, gene conversion). Segmental duplications can occur for genes with highly homologous gene family members or pseudogenes. High sequence similarity of a gene and homologous gene family member or pseudogene can lead to poor read alignments and variant calling. There is a need to informatically identify variants of genes with highly homologous gene family members or pseudogenes.

### SUMMARY

Disclosed herein include methods for determining GBA status. In some embodiments, a method for determining GBA status is under control of a processor (such as a hardware processor or a virtual processor) and comprises: receiving a first plurality of sequence reads generated from a sample obtained from a subject. The method can comprise: aligning the first plurality of sequence reads to a reference genome sequence to obtain a second plurality of sequence reads aligned to GBA gene or GBAP1 gene in the reference genome sequence. The method can comprise: determining a number of the sequence reads of the second plurality of sequence reads aligned to a unique region between GBA gene and GBAP1 gene in the reference genome sequence. The method can comprise: determining a normalized number of the sequence reads aligned to the unique region between GBA gene and GBAP1 gene in the reference genome sequence. The method can comprise: determining a total copy number of GBA gene and GBAP1 gene using a Gaussian mixture model comprising a plurality of Gaussians each representing a different integer copy number, given the normalized number of the sequence reads aligned to the region between GBA gene and GBAP1 gene. The method can comprise: phasing one or more haplotypes originating from GBA gene or GBAP1 gene in a region of GBA gene, or a corresponding region of GBAP1 gene, comprising a plurality of GBA/GBAP1 differentiating bases using sequence reads of the second plurality of sequence reads aligned to the region, or the corresponding region, comprising the plurality of GBA/GBAP1 differentiating bases. The method can comprise: determining a copy number of each of the one or more haplotypes using the total copy number of GBA gene and GBAP1 gene and a number of sequence reads of the second plurality of sequence reads each comprising one or more of the plurality of GBA/GBAP1 differentiating bases that support the haplotype. The method can comprise: determining a GBA status of the subject using the one or more haplotypes originating from GBA gene or GBAP1 gene in the region of GBA gene, or the corresponding region of GBAP1 gene, and/or the copy number of each of the one or more haplotypes. In some embodiments, the method comprises generating a user interface (UI) comprising a UI element representing or comprising the GBA status.

In some embodiments, the unique region between GBA gene and GBAP1 gene in the reference genome sequence comprises a unique region about 10 kilobases in length. The unique region between GBA gene and GBAP1 gene in the reference genome sequence can comprise chr1:155220429-155230539 of hg38 or a corresponding region of a reference human genome sequence.

In some embodiments, determining the normalized number of the sequence reads aligned to the unique region between GBA gene and GBAP1 gene in the reference genome sequence comprises: determining the normalized number of the sequence reads aligned to the unique region between GBA gene and GBAP1 gene in the reference genome sequence using (1a) a depth of the sequence reads aligned to the unique region between the GBA gene and GBAP1 gene, (1b) a length of the unique region, (2a) a depth of sequence reads of the first plurality of sequence reads aligned to each of a plurality of regions of the reference genome sequence other than a genetic locus comprising GBA gene and GBAP1 gene, and (2b) a length of each of the plurality of regions of the reference genome other than the genetic locus comprising GBA gene and GBAP1 gene.

In some embodiments, the method comprises: determining a normalized, corrected number of the sequence reads aligned to the unique region between GBA gene and GBAP1 gene in the reference genome sequence from the normalized number of the sequence reads aligned to the unique region between GBA gene and GBAP1 gene in the reference genome sequence. Determining the normalized, corrected number of the sequence reads aligned to the unique region between GBA gene and GBAP1 gene in the reference genome sequence can comprise: determining a normalized, GC content-corrected number of the sequence reads aligned to the unique region between GBA gene and GBAP1 gene in the reference genome sequence from the normalized number of the sequence reads aligned to the unique region between GBA gene and GBAP1 gene in the reference genome sequence. Determining the normalized, GC content-corrected number of the sequence reads aligned to the unique region between GBA gene and GBAP1 gene in the reference genome sequence can comprise: determining the normalized, GC content-corrected number of the sequence reads aligned to the unique region between GBA gene and GBAP1 gene in the reference genome sequence from the normalized number of the sequence reads aligned to the unique region between GBA gene and GBAP1 gene in the reference genome sequence using (1) a GC content of the unique region between the GBA gene and GBAP1 gene and optionally (2) a GC content of each of one or more regions of the reference genome sequence other than a genetic locus comprising GBA gene and GBAP1 gene. Determining the total copy number of GBA gene and GBAP1 gene can comprise: determining the total copy number of GBA gene and GBAP1 gene using the Gaussian mixture model, given the normalized, corrected number of the sequence reads aligned to the region between GBA gene and GBAP1 gene. In some embodiments, the method comprises: generating a user interface (UI) comprising a UI element representing or comprising the CYP21A2 status.

In some embodiments, determining the total copy number of GBA gene and GBAP1 gene comprises: determining a copy number of the region between GBA gene and GBAP1 gene using the Gaussian mixture model, given the normalized number of the sequence reads aligned to the region between GBA gene and GBAP1 gene. The total copy number of GBA gene and GBAP1 gene is the copy number of the region between GBA gene and GBAP1 gene plus two.

In some embodiments, determining the total copy number of GBA gene and

GBAP1 gene comprises: determining the total copy number of GBA gene and GBAP1 gene using a Gaussian mixture model and a predetermined posterior probability threshold, given the normalized number of the sequence reads aligned to the region between GBA gene and GBAP1 gene. The predetermined posterior probability threshold can be 0.95.

In some embodiments, the Gaussian mixture model comprises a one-dimensional Gaussian mixture model. The plurality of Gaussians of the Gaussian mixture model can represent integer copy numbers 0 to 10. The plurality of Gaussians of the Gaussian mixture model can comprise 5 Gaussians. A mean of each of the plurality of Gaussians can be the integer copy number represented by the Gaussian.

In some embodiments, phasing the one or more haplotypes originating from GBA gene or GBAP1 gene comprises: analyzing linkage information between GBA/GBAP1 differentiating bases of the plurality of GBA/GBAP1 differentiating bases using sequence reads of the second plurality of sequence reads aligned to the region, or the corresponding region, comprising the plurality of GBA/GBAP1 differentiating bases. Phasing the one or more haplotypes originating from GBA gene or GBAP1 gene can comprise: phasing the one or more haplotypes originating from GBA gene or GBAP1 gene using sequence reads of the second plurality of sequence reads each aligned two or more of the plurality of GBA/GBAP1 differentiating bases.

In some embodiments, a sequence read of the second plurality of sequence reads is aligned to the region of GBA gene, or the corresponding region of GBAP1 gene, comprising the plurality of GBA/GBAP1 differentiating bases with an alignment quality score of zero or more.

In some embodiments, the region of GBA gene, or the corresponding region of GBAP1 gene, comprising the plurality of GBA/GBAP1 differentiating bases is about 1.1 kilobases in length. The region of GBA gene, or the corresponding region of GBAP1 gene, comprising the plurality of GBA/GBAP1 differentiating bases can comprise exons 9-11 of GBA gene, or GBAP1 gene, respectively. The region of GBA gene, or the corresponding region of GBAP1 gene, comprising the plurality of GBA/GBAP1 differentiating bases can comprise p.L483P, p.D448H, c.1263del, RecNciI, RecTL, and c.1263del+RecTL. The plurality of GBA/GBAP1 differentiating bases can comprise 10 GBA/GBAP1 differentiating bases.

In some embodiments, the one or more haplotypes comprises a wildtype GBA haplotype, a wildtype GBAP1 haplotype, and/or a GBA/GBAP1 hybrid haplotype. The GBA/GBAP1 hybrid haplotype can comprise a GBA variant haplotype or a GBAP1 variant haplotype.

In some embodiments, determining the copy number of each of the one or more haplotypes comprises: determining a likelihood of one copy of a wildtype GBA haplotype is higher than a likelihood of two copies of the wildtype GBA haplotype given the number of sequence reads of the second plurality of sequence reads each comprising one or more of the plurality of GBA/GBAP1 differentiating bases that support the wildtype GBA haplotype. Determining the copy number of each of the one or more haplotypes can comprise: determining the copy number of the wildtype GBA haplotype is one. Determining the likelihood of one copy of the wildtype GBA haplotype is higher than the likelihood of two copies of the wildtype GBA haplotype can comprise: for each of one or more pairs (or all pairs) of consecutive GBA/GBAP1 differentiating bases of the plurality of GBA/GBAP1 differentiating bases for which a first haplotype of the one or more haplotypes comprises GBA bases at the consecutive GBA/GBAP1 differentiating bases and a second haplotype of the one or more haplotypes comprises a GBA base and a GBAP1 base, or a GBAP1 base and a GBA base, at the consecutive GBA/GBAP1 differentiating bases, determining the likelihood of one copy of the wildtype GBA haplotype is higher than the likelihood of two copies of the wildtype GBA haplotype given (1) a number of sequence reads of the second plurality of sequence reads each comprising the GBA bases at the consecutive GBA/GBAP1 differentiating bases, (2) a number of sequence reads of the second plurality of sequence reads each comprising the GBA base and the GBAP1 base at the consecutive GBA/GBAP1 differentiating bases, (3) a number of sequence reads of the second plurality of sequence reads each comprising the GBAP1 base and the GBA base, or the GBA base and the GBAP1 base at the consecutive GBA/GBAP1 differentiating bases, and/or (4) a number of sequence reads of the second plurality of sequence reads each comprising the GBAP1 bases at the consecutive GBA/GBAP1 differentiating bases. The likelihood of one copy of the wildtype GBA haplotype can comprise an aggregate (e.g., a weighted average or unweighted average) of the likelihood of one copy of the wildtype GBA haplotype determined for each of the one or more pairs of consecutive GBA/GBAP1 differentiating bases. The likelihood of two copies of the wildtype GBA haplotype can comprise an aggregate (e.g., a weighted average or unweighted average) of the likelihood of two copies of the wildtype GBA haplotype determined for each of the one or more pairs of consecutive GBA/GBAP1 differentiating bases

In some embodiments, the copy number of the wildtype GBA haplotype is one. Determining the GBA status of the subject can comprise: determining the subject is a carrier of a GBA variant haplotype. In some embodiments, the one or more haplotypes comprises four haplotypes. The total copy number of GBA gene and GBAP1 gene can be four. The copy number of each of the four haplotypes can be one. Determining the GBA status of the subject can comprise: determining the subject is a carrier of a GBA variant haplotype.

In some embodiments, the one or more haplotypes comprises two or more GBA variant haplotypes. None of the two or more GBA variant haplotypes can comprise a GBA base at each of the plurality of GBA/GBAP1 differentiating bases. None of the two or more GBA variant haplotypes can comprise GBA bases at all of the plurality of GBA/GBAP1 differentiating bases. Determining the GBA status of the subject can comprise: determining the subject is compound heterozygous of GBA variant haplotypes.

In some embodiments, the method comprises: determining a copy number of a GBA base at each of one or more of the plurality of GBA/GBAP1 differentiating bases is zero using sequence reads of the second plurality of sequence reads each comprising a base at the GBA/GBAP1 differentiating base that is not the GBA base. The base at the GBA/GBAP1 differentiating base that is not the GBA base is a GBAP1 base. Determining the GBA status can comprise: determining the subject is homozygous of each of the one or more of the plurality of GBAIGBAP/differentiating bases.

In some embodiments, the first plurality of sequence reads comprises sequence reads that are about 100 base pairs to about 1000 base pairs in length each. In some embodiments, the first plurality of sequence reads comprises paired-end sequence reads and/or single-end sequence reads. In some embodiments, the first plurality of sequence reads is generated by whole genome sequencing (WGS). The WGS can be clinical WGS (cWGS). In some embodiments, the sample comprises cells, cell-free DNA, cell-free fetal DNA, amniotic fluid, a blood sample, a biopsy sample, or a combination thereof.

Disclosed herein include methods of for determining CYP21A2 status. In some embodiments, a method for determining CYP21A2 status is under control of a processor (such as a hardware processor or a virtual processor) and comprises: receiving a first plurality of sequence reads generated from a sample obtained from a subject. The method can comprise: aligning the first plurality of sequence reads to a reference genome sequence to obtain a second plurality of sequence reads aligned to CYP21A2 gene or CYP21A1P pseudogene in the reference genome sequence. The method can comprise: determining a number of the sequence reads of the second plurality of sequence reads aligned to CYP21A2 gene or CYP21A1P pseudogene in the reference genome sequence. The method can comprise: determining a normalized number of the sequence reads aligned to CYP21A2 gene or CYP21A1P pseudogene in the reference genome sequence. The method can comprise: determining a total copy number of CYP21A2 gene and CYP21A1P pseudogene using a Gaussian mixture model comprising a plurality of Gaussians each representing a different integer copy number, given the normalized number of the sequence reads aligned to CYP21A2 gene or CYP21A1P pseudogene. The method can comprise: phasing one or more haplotypes originating from CYP21A2 gene or CYP21A1P pseudogene in a region of CYP21A2 gene, or a corresponding region of CYP21A1P pseudogene, comprising a plurality of CYP21A2/CYP21A1P differentiating bases using sequence reads of the second plurality of sequence reads aligned to the region, or the corresponding region, comprising the plurality of CYP21A2/CYP21A1P differentiating bases. The method can comprise: determining a copy number of each of the one or more haplotypes using the total copy number of CYP21A2 gene and CYP21A1P pseudogene and a number of sequence reads of the second plurality of sequence reads each comprising one or more of the plurality of CYP21A2/CYP21A1P differentiating bases that support the haplotype. The method can comprise: determining a CYP21A2 status of the subject using the one or more haplotypes originating from CYP21A2 gene or CYP21A1P pseudogene in the region of CYP21A2 gene, or the corresponding region of CYP21A1P pseudogene, and/or the copy number of each of the one or more haplotypes.

In some embodiments, determining the normalized number of the sequence reads aligned to CYP21A2 gene or CYP21A1P pseudogene in the reference genome sequence comprises: determining the normalized number of the sequence reads aligned to CYP21A2 gene or CYP21A1P pseudogene in the reference genome sequence using (1a) a depth of the sequence reads aligned to CYP21A2 gene or CYP21A1P pseudogene, (1b) a length of the unique region, (2a) a depth of sequence reads of the first plurality of sequence reads aligned to each of a plurality of regions of the reference genome sequence other than a genetic locus comprising CYP21A2 gene and CYP21A1P pseudogene, and (2b) a length of each of the plurality of regions of the reference genome other than the genetic locus comprising CYP21A2 gene and CYP21A1P pseudogene.

In some embodiments, the method comprises: determining a normalized, corrected number of the sequence reads aligned to CYP21A2 gene or CYP21A1P pseudogene in the reference genome sequence from the normalized number of the sequence reads aligned to CYP21A2 gene or CYP21A1P pseudogene in the reference genome sequence. Determining the normalized, corrected number of the sequence reads aligned to CYP21A2 gene or CYP21A1P pseudogene in the reference genome sequence can comprise: determining a normalized, GC content-corrected number of the sequence reads aligned to CYP21A2 gene or CYP21A1P pseudogene in the reference genome sequence from the normalized number of the sequence reads aligned to CYP21A2 gene or CYP21A1P pseudogene in the reference genome sequence. Determining the normalized, GC content-corrected number of the sequence reads aligned to CYP21A2 gene or CYP21A1P pseudogene in the reference genome sequence can comprise: determining the normalized, GC content-corrected number of the sequence reads aligned to CYP21A2 gene or CYP21A1P pseudogene in the reference genome sequence from the normalized number of the sequence reads aligned to CYP21A2 gene or CYP21A1P pseudogene in the reference genome sequence using (1) a GC content of CYP21A2 gene or CYP21A1P pseudogene and optionally (2) a GC content of each of one or more regions of the reference genome sequence other than a genetic locus comprising CYP21A2 gene and CYP21A1P pseudogene. Determining the total copy number of CYP21A2 gene and CYP21A1P pseudogene can comprise: determining the total copy number of CYP21A2 gene and CYP21A1P pseudogene using the Gaussian mixture model, given the normalized, corrected number of the sequence reads aligned to CYP21A2 gene or CYP21A1P pseudogene.

In some embodiments, determining the total copy number of CYP21A2 gene and CYP21A1P pseudogene comprises: determining the total copy number of CYP21A2 gene and CYP21A1P pseudogene using a Gaussian mixture model and a predetermined posterior probability threshold, given the normalized number of the sequence reads aligned to CYP21A2 gene or CYP21A1P pseudogene. The predetermined posterior probability threshold can be 0.95.

In some embodiments, the Gaussian mixture model comprises a one-dimensional Gaussian mixture model. The plurality of Gaussians of the Gaussian mixture model can represent integer copy numbers 0 to 10. The plurality of Gaussians of the Gaussian mixture model can comprise 5 Gaussians. A mean of each of the plurality of Gaussians can be the integer copy number represented by the Gaussian.

In some embodiments, phasing the one or more haplotypes originating from CYP21A2 gene or CYP21A1P pseudogene comprises: analyzing linkage information between CYP21A2/CYP21A1P differentiating bases of the plurality of CYP21A2/CYP21A1P differentiating bases using sequence reads of the second plurality of sequence reads aligned to the region, or the corresponding region, comprising the plurality of CYP21A2/CYP21A1P differentiating bases. Phasing the one or more haplotypes originating from CYP21A2 gene or CYP21A1P pseudogene comprises: phasing the one or more haplotypes originating from CYP21A2 gene or CYP21A1P pseudogene using sequence reads of the second plurality of sequence reads each aligned two or more of the plurality of CYP21A2/CYP21A1P differentiating bases.

In some embodiments, a sequence read of the second plurality of sequence reads is aligned to the region of CYP21A2 gene, or the corresponding region of CYP21A1P pseudogene, comprising the plurality of CYP21A2/CYP21A1P differentiating bases with an alignment quality score of zero or more.

In some embodiments, the plurality of CYP21A2/CYP21A1P differentiating bases comprises 14 CYP21A2/CYP21A1P differentiating bases. The 14 CYP21A2/CYP21A1P differentiating bases can comprise nine CYP21A2/CYP21A1P recombinant variants. The 14 CYP21A2/CYP21A1P differentiating bases can comprise chr6:32039081/32006353, 32039128/32006400, 32039132/32006404, 32039143/32006407, 32039426/32006690, 32039548/32006812, 32039802/32007066, 32039807/32007071, 32039810/32007074, 32039816/32007080, 32040182/32007446, 32040216/32007481, 32040421/32007686, and 32040535/32007800 of hg38, or corresponding bases thereof of a reference human genome sequence.

In some embodiments, the one or more haplotypes comprises a wildtype CYP21A2 haplotype, a wildtype CYP21A1P, and/or a CYP21A2/CYP21A1P hybrid haplotype. The CYP21A2/CYP21A1P hybrid haplotype can comprise a CYP21A2 variant haplotype or a CYP21A1P variant haplotype.

In some embodiments, determining the copy number of each of the one or more haplotypes comprises: determining a likelihood of one copy of a wildtype CYP21A2 haplotype is higher than a likelihood of two copies of the wildtype CYP21A2 haplotype given the number of sequence reads of the second plurality of sequence reads each comprising one or more of the plurality of CYP21A2/CYP21A1P differentiating bases that support the wildtype CYP21A2 haplotype. Determining the copy number of each of the one or more haplotypes can comprise: determining the copy number of the wildtype CYP21A2 haplotype is one. In some embodiments, determining the likelihood of one copy of the wildtype CYP21A2 haplotype is higher than the likelihood of two copies of the wildtype CYP21A2 haplotype comprises: for each of one or more pairs (or all pairs) of consecutive CYP21A2/CYP21A1P differentiating bases of the plurality of CYP21A2/CYP21A1P differentiating bases for which a first haplotype of the one or more haplotypes comprises CYP21A2 bases at the consecutive CYP21A2/CYP21A1P differentiating bases and a second haplotype of the one or more haplotypes comprises a CYP21A2 base and a CYP21A1P base, or a CYP21A1P base and a CYP21A2 base, at the consecutive CYP21A2/CYP21A1P differentiating bases, determining the likelihood of one copy of the wildtype CYP21A2 haplotype is higher than the likelihood of two copies of the wildtype CYP21A2 haplotype given (1) a number of sequence reads of the second plurality of sequence reads each comprising the CYP21A2 bases at the consecutive CYP21A2/CYP21A1P differentiating bases, (2) a number of sequence reads of the second plurality of sequence reads each comprising the CYP21A2 base and the CYP21A1P base at the consecutive CYP21A2/CYP21A1P differentiating bases, (3) a number of sequence reads of the second plurality of sequence reads each comprising the CYP21A1P base and the CYP21A2 base at the consecutive CYP21A2/CYP21A1P differentiating bases, and/or (4) a number of sequence reads of the second plurality of sequence reads each comprising the CYP21A1P bases at the consecutive CYP21A2/CYP21A1P differentiating bases. The likelihood of one copy of the wildtype CYP21A2 haplotype can comprise an aggregate (e.g., weighted average or unweighted average) of the likelihood of one copy of the wildtype CYP21A2 haplotype determined for each the one or more pairs of consecutive CYP21A2/CYP21A1P differentiating bases. The likelihood of two copies of the wildtype CYP21A2 haplotype can comprise an aggregate (e.g., weighted average or unweighted average) of the likelihood of two copies of the wildtype CYP21A2 haplotype determined for each the one or more pairs of consecutive CYP21A2/CYP21A1P differentiating bases.

In some embodiments, the copy number of the wildtype CYP21A2 haplotype is one. The method can comprise: determining the subject is a carrier of a CYP21A2 variant haplotype. In some embodiments, the one or more haplotypes comprises four haplotypes. The total copy number of CYP21A2 gene and CYP21A1P gene can be four. The copy number of each of the four haplotypes can be one. Determining the CYP21A2 status of the subject can comprise: determining the subject is a carrier of a CYP21A2 variant haplotype.

In some embodiments, the one or more haplotypes comprises two or more haplotypes. None of the two or more haplotypes may comprise CYP21A2 base at each of the plurality of CYP21A2/CYP21A1P differentiating bases. Each of the two or more haplotypes may not comprise CYP21A2 bases at all of the plurality of CYP21A2/CYP21A1P differentiating bases. The method can comprise: determining the subject is a compound heterozygous of CYP21A2 variant haplotypes.

In some embodiments, the one or more haplotypes comprises only one haplotype. The only one haplotype can comprise no CYP21A2 base at each of the plurality of CYP21A2/CYP21A1P differentiating bases. The method can comprise: determining the subject is homozygous of a CYP21A2 variant haplotype.

In some embodiments, the first plurality of sequence reads comprises sequence reads that are about 100 base pairs to about 1000 base pairs in length each. In some embodiments, the first plurality of sequence reads comprises paired-end sequence reads and/or single-end sequence reads. In some embodiments, the first plurality of sequence reads is generated by whole genome sequencing (WGS). The WGS can be clinical WGS (cWGS). In some embodiments, the sample comprises cells, cell-free DNA, cell-free fetal DNA, amniotic fluid, a blood sample, a biopsy sample, or a combination thereof.

Disclosed herein include systems (e.g., computing systems) for determining a gene recombinant variant. In some embodiments, a system for determining a gene recombinant variant comprises: non-transitory memory configured to store executable instructions and a first plurality of sequence reads generated from a sample obtained from a subject. The system can include: a processor such as a hardware processor or a virtual processor in communication with the non-transitory memory. The processor can be programmed by the executable instructions to perform: aligning the first plurality of sequence reads to a reference sequence to obtain a second plurality of sequence reads aligned to a gene or a gene paralog, or a region therebetween, in the reference sequence. The processor can be programmed by the executable instructions to perform: determining a total copy number of the gene and the gene paralog using a Gaussian mixture model comprising a plurality of Gaussians each representing a different integer copy number, given a number of the sequence reads aligned to the gene or the gene paralog, or a region therebetween. The processor can be programmed by the executable instructions to perform: phasing one or more haplotypes originating from the gene, comprising a recombinant variant of the gene, or the gene paralog, or a region of the gene or a corresponding region of the gene paralog, comprising a plurality of gene/gene paralog differentiating bases using sequence reads of the second plurality of sequence reads aligned to the region, or the corresponding region, comprising the plurality of gene/gene paralog differentiating bases. The processor can be programmed by the executable instructions to perform: determining a copy number of each of the one or more haplotypes using the total copy number of the gene and the gene paralog and a number of sequence reads of the second plurality of sequence reads each comprising one or more of the plurality of gene/gene paralog differentiating bases that support the haplotype.

In some embodiments, the gene recombinant variant comprises a reciprocal recombinant variant. The gene recombinant variant can comprise a non-reciprocal recombinant variant. In some embodiments, the reference sequence comprises a reference genome sequence.

In some embodiments, the processor is programmed by the executable instructions to perform: determining a number of the sequence reads aligned to the gene or the gene paralog, or a region therebetween. In some embodiments, the number of the sequence reads aligned to the gene or the gene paralog, or a region therebetween comprises a normalized and/or GC corrected number of the sequence reads aligned to the gene or the gene paralog, or a region therebetween. In some embodiments, the gene paralog is a gene. The gene paralog can be a pseudogene. In some embodiments, the gene and the gene paralog have a sequence identity of at least 90%.

In some embodiments, the gene is GBA gene, and the gene paralog is GBAP1 gene. In some embodiments, the gene is CYP21A2 gene, and the gene paralog is CYP21A1P pesudogene. In some embodiments, the gene is ABCC6, ABCD1, ACTB, ACTG1, ACTN4, ADAMTSL2, ADIPOR1, AFG3L2, AGK, ALG1, ALMS1, ANKRD11, ANOS1, AP4S1, ARSE, ASNS, ATAD3A, B3GAT3, BCAP31, BDP1, BMPR1A, BRAF, BRCA1, C2, CACNA1C, CALM1, CD46, CEP290, CFH, CFH, CFH, CHEK2, CISD2, CLCNKA, CLCNKB, CORO1A, COX10, CP, CRYBB2, CSF2RA, CUBN, CUBN, CYCS, CYP11B1, CYP21A2, DCLRE1C, DHFR, DICER1, DIS3L2, DNAH1, DNAH1, DNM1, DSE, DUOX2, EGLN1, ELK1, ELMO2, ERCC6, ESPN, EYS, F8, FANCD2, FANCD2, FAR1, FHL1, FLG, FLNC, FOXD4, FXN, GBA, GH1, GK, GOSR2, GUSB, HBA1, HBA2, HNRNPA1, HPS1, HSPD1, HYD1N, IDS, IFT122, KANSL1, KCTD1, KIF1C, KRAS, KRTI4, KRTI6, KRT17, KRT6A, KRT6B, KRT6C, LEFTY2, LRP5, LRP5, MAT2A, MID1, MOCS1, MSN, MSX2, MYO5B, NCF1, NEB, NECAP1, NEFH, NF1, NF1, NF1, NOTCH2, NXF5, OCLN, OTOA, PARN, PBX1, PIGA, PIGN, PIK3CA, PIK3CD, PKD1, PKP2, PMS2, PMS2, PMS2, PNPT1, POLH, PRODH, PRODH, PROS1, PRPS1, PRSS1, PTEN, RAD21, RBM8A, RBPJ, RDX, RMND1, RNF216, RNF216, RPL15, SALL1, SBDS, SDHA, SHOX, SLC25A15, SLC25A15, SLC33A1, SLC6A8, SMN1, SMN2, SOX2, SPTLC1, SRD5A3, SRP72, STAT5B, STRC, SYT14, TARDBP, TBL1XR1, TBX20, TMM8A, TPM3, TPMT, TRAPPC2, TRIP11, TTN, TUBA1A, TUBB2A, TUBB2B, TUBB3, TUBB4A, TUBG1, TYR, UBA5, UBE3A, UNC93B1, USP18, VPS35, VWF, WRN, XIAP, ZEB2, or ZNF341.

In some embodiments, the processor is programmed by the executable instructions to perform: determining a gene variant status of the subject using the one or more haplotypes originating from the gene or the gene paralog, or the region of the gene or the corresponding region of the gene paralog, and/or the copy number of each of the one or more haplotypes. In some embodiments, the processor can be programmed by the executable instructions to perform: generating a user interface (UI) comprising a UI element representing or comprising the gene variant status.

In some embodiments, determining the normalized number of the sequence reads aligned to the gene or the gene paralog in the reference sequence comprises: determining the normalized number of the sequence reads aligned to the gene or the gene paralog in the reference sequence using (1a) a depth of the sequence reads aligned to the gene or the gene paralog, (1b) a length of the unique region, (2a) a depth of sequence reads of the first plurality of sequence reads aligned to each of a plurality of regions of the reference sequence other than a genetic locus comprising the gene and the gene paralog, and (2b) a length of each of the plurality of regions of the reference sequence other than the genetic locus comprising the gene and the gene paralog.

In some embodiments, the processor is programmed by the executable instructions to perform: determining a normalized, corrected number of the sequence reads aligned to the gene or the gene paralog in the reference sequence from the normalized number of the sequence reads aligned to the gene or the gene paralog in the reference sequence. Determining the normalized, corrected number of the sequence reads aligned to the gene or the gene paralog in the reference sequence can comprise: determining a normalized, GC content-corrected number of the sequence reads aligned to the gene or the gene paralog in the reference sequence from the normalized number of the sequence reads aligned to the gene or the gene paralog in the reference sequence. Determining the normalized, GC content-corrected number of the sequence reads aligned to the gene or the gene paralog in the reference sequence can comprise: determining the normalized, GC content-corrected number of the sequence reads aligned to the gene or the gene paralog in the reference sequence from the normalized number of the sequence reads aligned to the gene or the gene paralog in the reference sequence using (1) a GC content of the gene or the gene paralog and optionally (2) a GC content of each of one or more regions of the reference sequence other than a genetic locus comprising the gene and the gene paralog. Determining the total copy number of the gene and the gene paralog can comprise: determining the total copy number of the gene and the gene paralog using the Gaussian mixture model, given the normalized, corrected number of the sequence reads aligned to the gene or the gene paralog.

In some embodiments, determining the total copy number of the gene and the gene paralog comprises: determining a copy number of a region between the gene or the gene paralog using the Gaussian mixture model, given the normalized number of the sequence reads aligned to the gene or the gene paralog. The total copy number of the gene and the gene paralog can be the copy number of the region between the gene or the gene paralog plus two.

In some embodiments, determining the total copy number of the gene and the gene paralog comprises: determining the total copy number of the gene and the gene paralog using a gaussian mixture model and a predetermined posterior probability threshold, given the normalized number of the sequence reads aligned to the gene or the gene paralog. The predetermined posterior probability threshold can be 0.95.

In some embodiments, the Gaussian mixture model comprises a one-dimensional Gaussian mixture model. The plurality of Gaussians of the Gaussian mixture model can represent integer copy numbers 0 to 10. The plurality of Gaussians of the Gaussian mixture model can comprise 5 Gaussians. A mean of each of the plurality of Gaussians can be the integer copy number represented by the Gaussian.

In some embodiments, phasing the one or more haplotypes originating from the gene or the gene paralog comprises: analyzing linkage information between gene/gene paralog differentiating bases of the plurality of the gene/gene paralog differentiating bases using sequence reads of the second plurality of sequence reads aligned to the region, or the corresponding region, comprising the plurality of gene/gene paralog differentiating bases. Phasing the one or more haplotypes originating from the gene or the gene paralog can comprise: phasing the one or more haplotypes originating from the gene or the gene paralog using sequence reads of the second plurality of sequence reads each aligned two or more of the plurality of gene/gene paralog differentiating bases. In some embodiments, a sequence read of the second plurality of sequence reads is aligned to the region of the gene, or the corresponding region of the gene paralog, comprising the plurality of gene/gene paralog differentiating bases with an alignment quality score of zero or more.

In some embodiments, the one or more haplotypes comprises a wildtype gene haplotype, a wildtype gene paralog, and/or a gene/gene paralog hybrid haplotype. The gene/gene paralog hybrid haplotype can comprise a gene variant haplotype or a gene paralog variant haplotype.

In some embodiments, determining the copy number of each of the one or more haplotypes can comprise: determining a likelihood of one copy of a wildtype gene haplotype is higher than a likelihood of two copies of the wildtype gene haplotype given the number of sequence reads of the second plurality of sequence reads each comprising one or more of the plurality of gene/gene paralog differentiating bases that support the wildtype gene haplotype. Determining the copy number of each of the one or more haplotypes can comprise: determining the copy number of the wildtype gene haplotype is one. Determining the likelihood of one copy of the wildtype gene haplotype is higher than the likelihood of two copies of the wildtype gene haplotype can comprise: for each of one or more pairs (or all pairs) of consecutive gene/gene paralog differentiating bases of plurality of gene/gene paralog differentiating bases for which a first haplotype of the one or more haplotypes comprises gene bases at the consecutive gene/gene paralog differentiating bases and a second haplotype of the one or more haplotypes comprises a gene base and a gene paralog base, or a gene paralog base and a gene base, at the consecutive gene/gene paralog differentiating bases, determining the likelihood of one copy of the wildtype gene haplotype is higher than the likelihood of two copies of the wildtype gene haplotype given (1) a number of sequence reads of the second plurality of sequence reads each comprising the gene bases at the consecutive gene/gene paralog differentiating bases, (2) a number of sequence reads of the second plurality of sequence reads each comprising the gene base and the gene paralog base at the consecutive gene/gene paralog differentiating bases, (3) a number of sequence reads of the second plurality of sequence reads each comprising the gene paralog base and the gene base at the consecutive gene/gene paralog differentiating bases, and/or (4) a number of sequence reads of the second plurality of sequence reads each comprising the gene paralog bases at the consecutive gene/gene paralog differentiating bases. The likelihood of one copy of the wildtype gene haplotype can comprise an aggregate (e.g., a weighted or unweighted average) of the likelihood of one copy of the wildtype gene haplotype determined for each of the one or more pairs of consecutive gene/gene paralog differentiating bases. The likelihood of two copies of the wildtype gene haplotype can comprise an aggregate (e.g., a weighted or unweighted average) of the likelihood of two copies of the wildtype gene haplotype determined for each of the one or more pairs of consecutive gene/gene paralog differentiating bases

In some embodiments, the copy number of the wildtype gene haplotype is one. The processor can be programmed by the executable instructions to perform: determining the subject is a carrier of a gene variant haplotype. In some embodiments, the one or more haplotypes comprises four haplotypes. The total copy number of the gene and the gene paralog can be four. The copy number of each of the four haplotypes can be one. Determining the gene variant status of the subject can comprise: determining the subject is a carrier of a gene variant haplotype.

In some embodiments, the one or more haplotypes comprises two or more haplotypes. None of the two or more haplotypes may comprise a gene base at each of the plurality of gene/gene paralog differentiating bases. Each of the two or more haplotypes may comprise no gene bases at all of the plurality of gene/gene paralog differentiating bases. The processor can be programmed by the executable instructions to perform: determining the subject is a compound heterozygous of gene variant haplotypes.

In some embodiments, the one or more haplotypes comprises only one haplotype. The only one haplotype can comprise no gene base at each of plurality of the gene/gene paralog differentiating bases. The processor can be programmed by the executable instructions to perform: determining the subject is homozygous of a gene variant haplotype.

In some embodiments, the first plurality of sequence reads comprises sequence reads that are about 100 base pairs to about 1000 base pairs in length each. In some embodiments, the first plurality of sequence reads comprises paired-end sequence reads and/or single-end sequence reads. In some embodiments, the first plurality of sequence reads is generated by whole genome sequencing (WGS). The WGS can be clinical WGS (cWGS). In some embodiments, the sample comprises cells, cell-free DNA, cell-free fetal DNA, amniotic fluid, a blood sample, a biopsy sample, or a combination thereof.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 illustrates analyzing the linkage information between a set of reliable base differences or sites between a gene and an analog of the gene provided by reads and read pairs.

FIGS. 2A1-2A2, 2B1-2B2, and 2C1-2C2 show non-limiting exemplary detection of challenging GBA variants through targeted copy number calling and haplotype phasing.

FIGS. 3A-3C show non-limiting exemplary detection of challenging CYP21A2 variants.

FIG. 4 is a flow diagram showing an exemplary method of determining or identifying one or more GBA variant or GBA variant status (e.g., carrier, compound heterozygous, or homozygous).

FIG. 5 is a flow diagram showing an exemplary method of determining or identifying one or more CYP21A2 variants or variant status (e.g., carrier, compound heterozygous, or homozygous).

FIG. 6 is a flow diagram showing an exemplary method of determining or identifying one or more gene recombinant variants or gene variant status (e.g., carrier, compound heterozygous, or homozygous).

FIG. 7 is a block diagram of an illustrative computing system configured to determine or identify one or more gene recombinant variants (e.g., GBA variants, CYP21A2 variants) or gene variant status (e.g., carrier, compound heterozygous, or homozygous).

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for determining GBA status. In some embodiments, a method for determining GBA status is under control of a processor (such as a hardware processor or a virtual processor) and comprises: receiving a first plurality of sequence reads generated from a sample obtained from a subject. The method can comprise: aligning the first plurality of sequence reads to a reference genome sequence to obtain a second plurality of sequence reads aligned to GBA gene or GBAP1 gene in the reference genome sequence. The method can comprise: determining a number of the sequence reads of the second plurality of sequence reads aligned to a unique region between GBA gene and GBAP1 gene in the reference genome sequence. The method can comprise: determining a normalized number of the sequence reads aligned to the unique region between GBA gene and GBAP1 gene in the reference genome sequence. The method can comprise: determining a total copy number of GBA gene and GBAP1 gene using a Gaussian mixture model comprising a plurality of Gaussians each representing a different integer copy number, given the normalized number of the sequence reads aligned to the region between GBA gene and GBAP1 gene. The method can comprise: phasing one or more haplotypes originating from GBA gene or GBAP1 gene in a region of GBA gene, or a corresponding region of GBAP1 gene, comprising a plurality of GBA/GBAP1 differentiating bases using sequence reads of the second plurality of sequence reads aligned to the region, or the corresponding region, comprising the plurality of GBA/GBAP1 differentiating bases. The method can comprise: determining a copy number of each of the one or more haplotypes using the total copy number of GBA gene and GBAP1 gene and a number of sequence reads of the second plurality of sequence reads each comprising one or more of the plurality of GBA/GBAP1 differentiating bases that support the haplotype. The method can comprise: determining a GBA status of the subject using the one or more haplotypes originating from GBA gene or GBAP1 gene in the region of GBA gene, or the corresponding region of GBAP1 gene, and/or the copy number of each of the one or more haplotypes.

Disclosed herein include methods of for determining CYP21A2 status. In some embodiments, a method for determining CYP21A2 status is under control of a processor (such as a hardware processor or a virtual processor) and comprises: receiving a first plurality of sequence reads generated from a sample obtained from a subject. The method can comprise: aligning the first plurality of sequence reads to a reference genome sequence to obtain a second plurality of sequence reads aligned to CYP21A2 gene or CYP21A1P pseudogene in the reference genome sequence. The method can comprise: determining a number of the sequence reads of the second plurality of sequence reads aligned to CYP21A2 gene or CYP21A1P pseudogene in the reference genome sequence. The method can comprise: determining a normalized number of the sequence reads aligned to CYP21A2 gene or CYP21A1P pseudogene in the reference genome sequence. The method can comprise: determining a total copy number of CYP21A2 gene and CYP21A1P pseudogene using a Gaussian mixture model comprising a plurality of Gaussians each representing a different integer copy number, given the normalized number of the sequence reads aligned to CYP21A2 gene or CYP21A1P pseudogene. The method can comprise: phasing one or more haplotypes originating from CYP21A2 gene or CYP21A1P pseudogene in a region of CYP21A2 gene, or a corresponding region of CYP21A1P pseudogene, comprising a plurality of CYP21A2/CYP21A1P differentiating bases using sequence reads of the second plurality of sequence reads aligned to the region, or the corresponding region, comprising the plurality of CYP21A2/CYP21A1P differentiating bases. The method can comprise: determining a copy number of each of the one or more haplotypes using the total copy number of CYP21A2 gene and CYP21A1P pseudogene and a number of sequence reads of the second plurality of sequence reads each comprising one or more of the plurality of CYP21A2/CYP21A1P differentiating bases that support the haplotype. The method can comprise: determining a CYP21A2 status of the subject using the one or more haplotypes originating from CYP21A2 gene or CYP21A1P pseudogene in the region of CYP21A2 gene, or the corresponding region of CYP21A1P pseudogene, and/or the copy number of each of the one or more haplotypes.

Disclosed herein include systems (e.g., computing systems) for determining a gene recombinant variant. In some embodiments, a system for determining a gene recombinant variant comprises: non-transitory memory configured to store executable instructions and a first plurality of sequence reads generated from a sample obtained from a subject. The system can include: a processor such as a hardware processor or a virtual processor in communication with the non-transitory memory. The processor can be programmed by the executable instructions to perform: aligning the first plurality of sequence reads to a reference sequence to obtain a second plurality of sequence reads aligned to a gene or a gene paralog, or a region therebetween, in the reference sequence. The processor can be programmed by the executable instructions to perform: determining a total copy number of the gene and the gene paralog using a Gaussian mixture model comprising a plurality of Gaussians each representing a different integer copy number, given a number of the sequence reads aligned to the gene or the gene paralog, or a region therebetween. The processor can be programmed by the executable instructions to perform: phasing one or more haplotypes originating from the gene, comprising a recombinant variant of the gene, or the gene paralog, or a region of the gene or a corresponding region of the gene paralog, comprising a plurality of gene/gene paralog differentiating bases using sequence reads of the second plurality of sequence reads aligned to the region, or the corresponding region, comprising the plurality of gene/gene paralog differentiating bases. The processor can be programmed by the executable instructions to perform: determining a copy number of each of the one or more haplotypes using the total copy number of the gene and the gene paralog and a number of sequence reads of the second plurality of sequence reads each comprising one or more of the plurality of gene/gene paralog differentiating bases that support the haplotype.

### Detecting of Variants in Short-Read Data

Segmental duplications are hotspots for structural variants (for example, with deletion or duplication) with gene recombinant variants (for example, gene conversion). A gene recombinant variant can result from a sequence of a gene being copied into a paralog of the gene or vice versa. The paralog of the gene can be a gene or a pseudogene. Segmental duplications can occur for genes with highly homologous gene family members or pseudogenes. Many clinically relevant genes have highly homologous gene family members or pseudogenes and can be affected by segmental duplications. Such clinically relevant genes include genes important for rare diseases, cancer, immunology and pharmacogenetics.

Analyzing sequence reads of genes that have undergone segmental duplications, such read alignments and variant calling, can be informatically challenging. Such analysis can require the combined assessment of different variants, including single nucleotide polymorphism (SNP), insertion and deletion (indel), copy number variation (CNV), and structural variation (SV). High sequence similarity of a gene and a homologous gene family member or pseudogene can lead to poor sequence read alignments and variant calling. Standard secondary analysis pipelines may yield no or unreliable results. For example, a gene and a paralog of the gene can differ by just a few bases (such as 2, 3, 4, 5, 6, 7, 8, 9, 10, 20, 30, or more bases), making alignment and variant calling difficult. When a gene and a paralog of the gene differ by a few bases, sequence reads that do not include such bases can be aligned to the gene or the paralog with the same or similar alignment scores (e.g., percentages of mismatches). Consequently, aligning reads to the gene and the paralog is difficult and results in low alignment quality (e.g., MapQ quality). As a result, variant calling using such low quality read alignments can be difficult.

Targeted CNV calling of the gene and the paralog of the gene can be useful. The total copy number of the gene and the paralog can be determined with read counting and normalization using all reads. The population depth distribution can be modeled to call copy numbers. The gene and the paralog of the gene can be differentiated and their copy numbers determined using sequence read counts at fixed base differences. Gene fusions can be determined based on, for example, a change in the copy number of the gene at fixed base differences. Targeted calling of SNPs and indels can be performed. Star alleles can be called based on all the called variants and assigned into haplotypes. One, some, a majority, or all of these processes can be performed serially by one or multiple computing systems or in parallel by multiple computing systems.

The methods disclosed herein can be used to detect short gene recombination, such as gene conversions, where the sequences of a gene are mutated to be identical to that of another gene (e.g., a paralog of the gene). Each sequence being mutated can be as small as a single base. The methods can enable detection of single base gene conversions by haplotype phasing. Gene recombinant variants (reciprocal or non-reciprocal) of GBA gene can result in Gaucher disease, which has a 1 in 50,000-100,000 incidence rate. Heterozygotes are associated with Parkinson's disease. Gene recombinant variant of CYP21A2 gene can result in 21-Hydroxylase-Deficient Congenital Adrenal Hyperplasia (21-OHD CAH), which has a 1:10,000-1:16,000 incidence rate.

The gene of interest and a paralog of the gene of interest (e.g., a pseudogene) are referred to herein as gene A and gene B. The presence of gene B in a reference genome to which sequence reads (e.g., short-read sequence reads) can lead to difficulty in alignment and variant calling. Often, a small stretch of sequence (or single base) of gene A can be mutated to look identical as the corresponding sequence in gene B by, for example, recombination, such as gene conversion. This type of recombinant or gene conversion variants can be extremely difficult to detect as the variant containing reads of gene A may align to gene B instead of gene A.

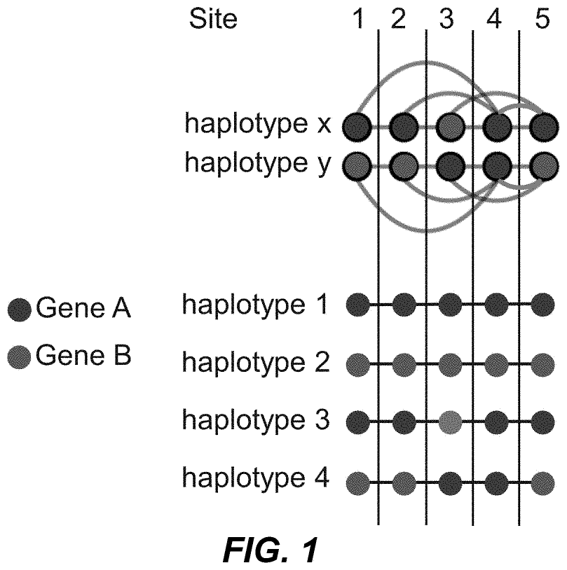

Calling carrier samples or compound heterozygotes. Haplotype phasing can be performed based on a set of reliable base differences or sites between gene A and gene B. Based on a set of reliable base differences or sites between gene A and gene B, a caller for calling or identifying recombinant or gene conversion variants can analyze the linkage information between these differences sites provided by reads and read pairs. Linkage information can be analyzed by, for example, read backed phasing. One read or read pair that covers Site n and Site m can indicate whether the haplotype the read or read pair originates from has gene A or B base at Site n and gene A or B base at Site m. Then, the caller can phase all the haplotypes originating from either gene A or gene B in the region with the set of reliable base differences and identify haplotypes of gene A and gene B and one or more hybrid haplotypes (a mixture of gene A and gene B bases on the same haplotype. Referring to FIG. 1, one read pair that covers Site 1 and Site 4 can indicate the haplotype (haplotype x) the read pair originates from has gene A base at Site 1 and gene A base at Site 4. One read pair that covers Site 3 and Site 5 can indicate the haplotype (haplotype y) the read pair originates from has gene A base at Site 3 and gene B base at Site 5. One read that covers Site 4 and Site 5 can indicate the haplotype (haplotype y) the read originates from has gene A base at Site 4 and gene B base at Site 5. The caller can phase all the haplotypes originating from either gene A or gene B in the region with the set of five reliable base differences and identify haplotypes of gene A (haplotype 1) and gene B (haplotype 2) and hybrid haplotypes (haplotypes 3 and 4). The number of haplotypes, and the number of sites, and the sites (e.g., Sites 1 and 4, or Sites 3 and 5) sequence reads cover are shown in FIG. 1 for illustration only and are not intended to be limiting.

To assess the relative abundance of the different haplotypes, the caller can use the total copy number (CN) of gene A and gene B as well as haplotype-supporting read counts at the differentiating bases to call CN of each haplotype. The total CN of gene A and gen B can be determined using a Gaussian mixture model. Determining CNs using Gaussian mixture models by a SMN caller and a CYP2D6 caller have been described in PCT Publication No. WO 2021/045947, entitled METHODS AND SYSTEMS FOR DIAGNOSING FROM WHOLE GENOME SEQUENCING DATA, the content of which is incorporated herein by reference in its entirety. The caller can compare two scenarios: one copy of the wildtype gene A haplotype vs. two copies of the wildtype gene A haplotype. The caller can determine which scenario is more likely given the number of supporting reads in the data. If the caller calls only one copy of the wildtype gene A haplotype, this indicates that the individual is a carrier of the disease-causing variant. If an individual is a carrier of more than one variant haplotype and there is no haplotype that carries the gene A base at all variant sites of interest, then the caller calls this sample as compound heterozygous with no copies of wildtype gene A.

Calling samples homozygous for a variant. Based on a list of gene conversion variants to detect, the caller can call the copy number (CN) of the gene A base. Using the number of reads supporting the gene A base or gene B base, as well as the total CN of gene A and gene B, the most likely combination of the CN of the gene A base and the CN of the gene B base can be determined. If the CN of the gene A base is called as 0, this indicates that the individual has no copy of the wildtype gene A (the haplotype that carries the gene A base at a variant site of interest), and is homozygous for the gene conversion variant.

### GBA Variants

### Detection of Challenging GBA Variants in Short-Read WGS Data

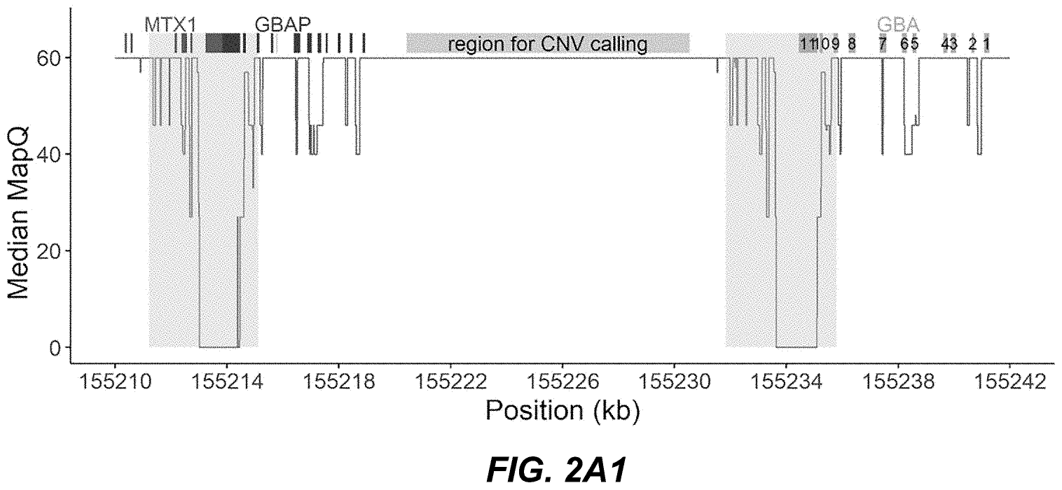

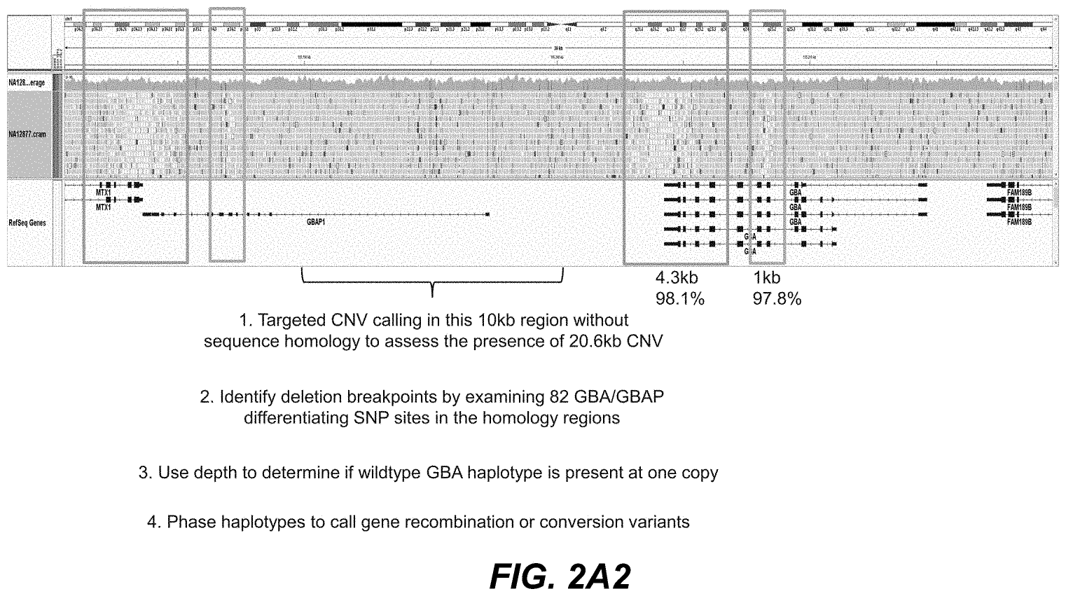

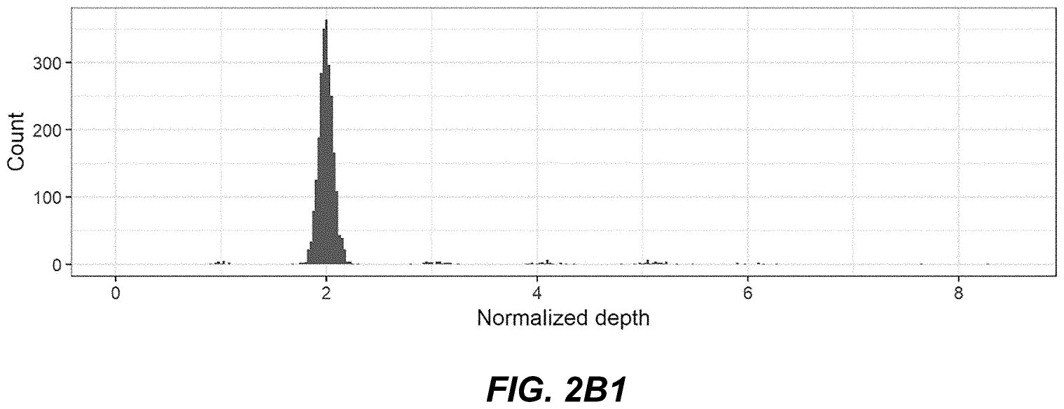

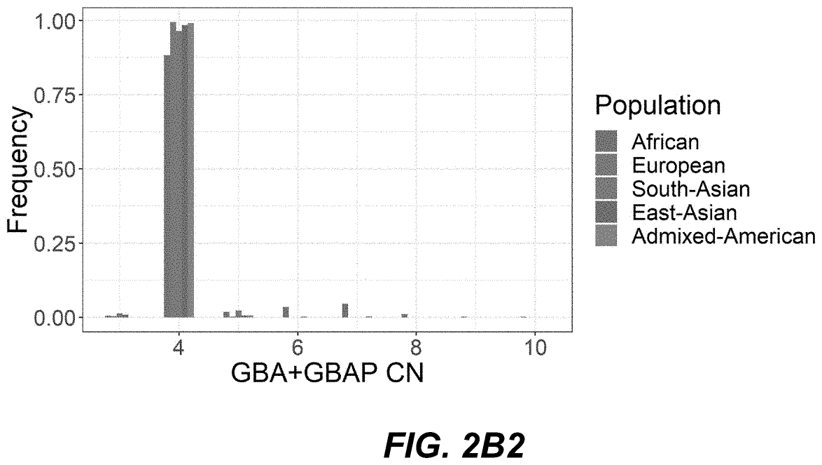

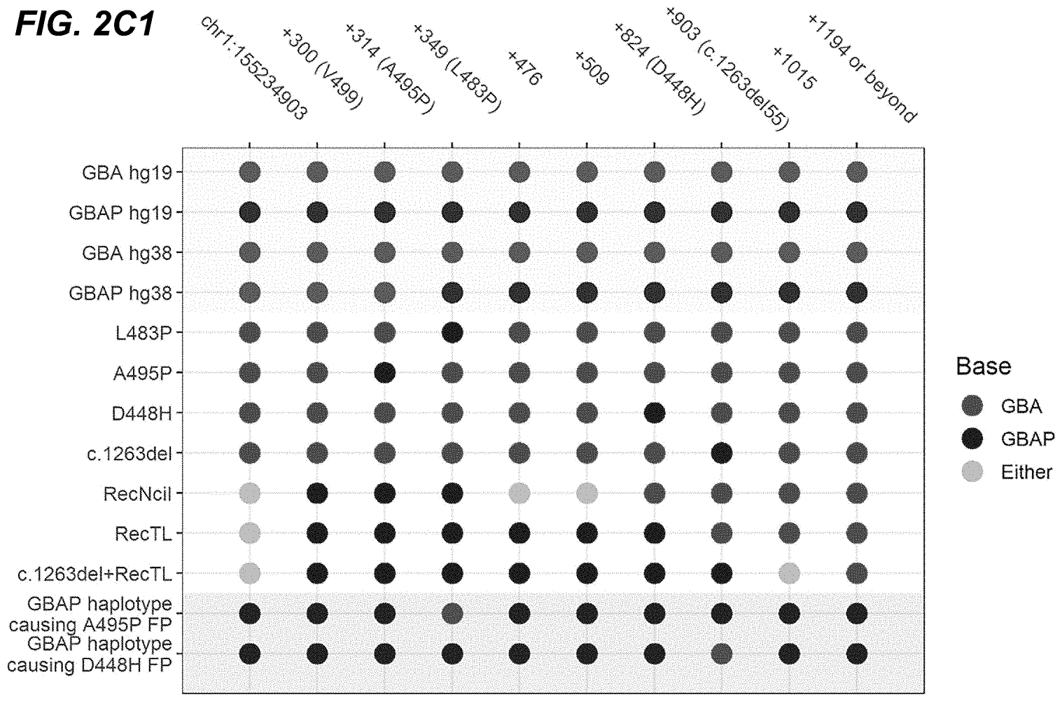

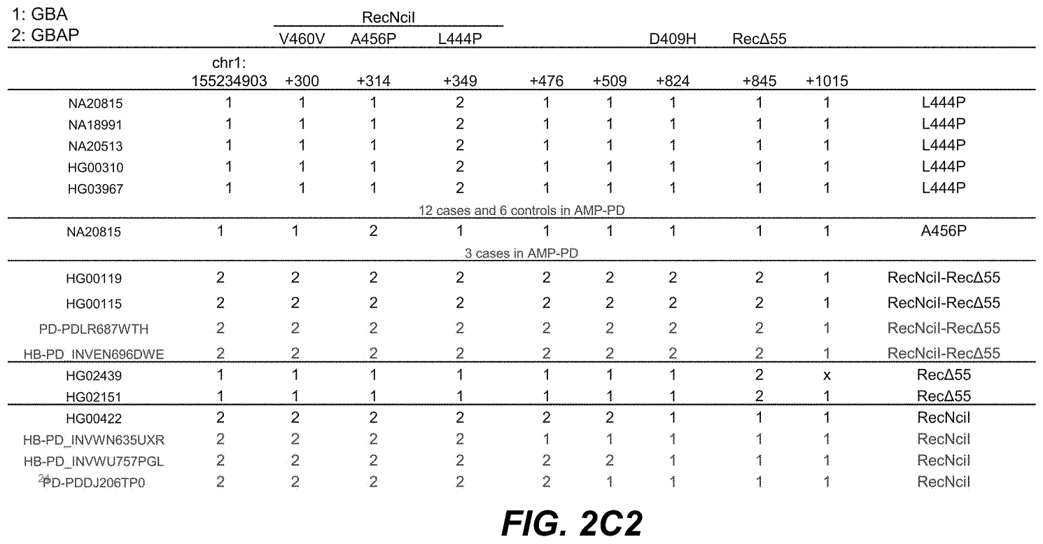

The sequence homology between GBA and GBAP1 leads to reduced mapping quality (FIG. 2A1) and less accurate variant calls by standard secondary analysis pipelines. In addition, the recombinant variants where the GBA base is mutated to the corresponding base in GBAP1 are challenging to detect as the variant reads align to GBAP1. Thus, using 2405 WGS data sets from the 1000 genome project (1 kGP), Gauchian, a novel WGS-based bioinformatics method to call GBA variants, was developed.

Gauchian analysis starts from determining copy number changes. Reciprocal recombination across homologous regions lead to copy number gains (duplications) or losses (fusions) of the 20.6 kb region between the two genes. Since the breakpoint may vary in position, Gauchian uses the sequencing depth in the unique region between the two genes to detect the copy number variants (CNVs) (FIGS. 2A1 2A2, 2B1, and 2B2). Out of 2504 1 kGP samples, 108 samples were found to carry reciprocal recombination (15 deletions and 93 duplications). Duplications may create GBA-GBAP1 merging, but always leave two intact copies of GBA, while fusions can create GBA variants (GBA-GBAP1 fusions) if the deletion breakpoint falls within the GBA gene. After determining copy number changes, Gauchian used the base differences between homologous regions of GBA and GBAP1 shown in Table 1 to differentiate the two genes and identify the breakpoints of the CNV (FIG. 2A2). The major homology region, Exon9-11, is where pathogenic deletions most likely happen. Therefore, Gauchian phased both GBA and GBAP1 haplotypes through the Exon9-11 homology region to further resolve breakpoints (FIGS. 2C1 and 2C2). For 1 kGP deletion samples, Gauchian identified breakpoints that do not alter the GBA gene in all but one sample. Breakpoints mostly fell in a region that is identical between GBA and GBAP1, extending from the 3′UTR to beyond the gene (the zero mapping quality region in FIG. 2A1). Such CNVs were known and leave the GBA gene, or at least the coding region, intact, so appear benign. In one deletion sample, Gauchian identified the breakpoint in Exon9-11, which creates a RecNciI fusion (FIGS. 2C1 and 2C2).

TABLE 1
Base differences between homologous regions of
GBA and GBAP1 used to differentiate the two
genes and identify the breakpoints of the CNV

| GBA | GBA | GBAP1 | GBAP1 |
|---|---|---|---|
| position | base | position | base |
| 155231496 | c | 155210868 | g |
| 155231522 | a | 155210894 | g |
| 155231557 | t | 155210929 | c |
| 155231692 | g | 155211064 | a |
| 155231693 | c | 155211065 | t |
| 155231701 | t | 155211073 | c |
| 155231816 | gaacacag | 155211188 | gaacag |
| 155231834 | g | 155211204 | a |
| 155231848 | a | 155211218 | c |
| 155231857 | t | 155211227 | c |
| 155231870 | a | 155211240 | g |
| 155231923 | t | 155211295 | a |
| 155231936 | a | 155211308 | g |
| 155231937 | g | 155211309 | a |
| 155231941 | a | 155211313 | g |
| 155231942 | g | 155211314 | a |
| 155231989 | g | 155211363 | a |
| 155232135 | t | 155211509 | a |
| 155232231 | g | 155211605 | a |
| 155232340 | c | 155211714 | t |
| 155232418 | a | 155211792 | g |
| 155232440 | t | 155211814 | c |
| 155232447 | a | 155211821 | g |
| 155232451 | a | 155211825 | g |
| 155232540 | g | 155211914 | c |
| 155232549 | c | 155211923 | g |
| 155232550 | g | 155211924 | a |
| 155232572 | t | 155211946 | c |
| 155232721 | c | 155212095 | t |
| 155232724 | c | 155212098 | t |
| 155232784 | a | 155212158 | g |
| 155232834 | a | 155212208 | c |
| 155232892 | g | 155212266 | t |
| 155232893 | a | 155212267 | t |
| 155232916 | g | 155212290 | c |
| 155232919 | a | 155212293 | g |
| 155232927 | g | 155212301 | a |
| 155232935 | a | 155212309 | g |
| 155232982 | g | 155212356 | a |
| 155233046 | a | 155212420 | g |
| 155233268 | c | 155212642 | g |
| 155233287 | g | 155212661 | a |
| 155233514 | a | 155212888 | g |
| 155233517 | c | 155212891 | t |
| 155233521 | t | 155212895 | a |
| 155233531 | g | 155212905 | a |
| 155233612 | a | 155212985 | g |
| 155233639 | g | 155213012 | a |
| 155234903 | c | 155214276 | t |
| 155235203 | c | 155214576 | g |
| 155235217 | c | 155214590 | g |
| 155235252 | a | 155214625 | g |
| 155235379 | a | 155214752 | g |
| 155235412 | g | 155214785 | a |
| 155235727 | c | 155215100 | g |
| 155235748 | tgggactg | 155215121 | tgggttcc |
| 155235803 | aaggttcc | 155215121 | tgggttcc |
| 155235918 | a | 155215236 | g |
| 155236097 | g | 155215415 | c |
| 155236102 | t | 155215420 | c |
| 155236129 | t | 155215447 | c |
| 155236145 | g | 155215463 | c |
| 155236175 | g | 155215493 | a |
| 155236190 | a | 155215508 | g |
| 155237982 | t | 155216705 | c |
| 155237989 | g | 155216712 | a |
| 155237990 | c | 155216713 | t |
| 155237996 | g | 155216719 | t |
| 155237997 | c | 155216720 | t |
| 155238055 | t | 155216778 | c |
| 155238088 | t | 155216811 | c |
| 155238092 | t | 155216815 | g |
| 155238141 | a | 155216864 | t |
| 155238174 | c | 155216897 | t |
| 155238192 | a | 155216915 | g |
| 155238206 | a | 155216929 | c |
| 155238214 | a | 155216937 | c |
| 155238630 | g | 155217353 | a |
| 155238754 | g | 155217477 | a |
| 155238871 | cagg | 155217594 | ctg |
| 155238882 | a | 155217604 | g |
| 155238929 | a | 155217651 | t |
| 155238930 | g | 155217652 | t |

In addition to CNVs arising from reciprocal recombination, Gauchian performed targeted calling of pathogenic GBA variants, including simple small variants as well as gene conversions and challenging SNVs in Exon9-11 where the GBA base is mutated to the corresponding base in GBAP1, presumably also arising through small gene conversion events. These include p.L483P, p.D448H, c.1263del (55 bp deletion), RecNciI (including the 3 SNVs p.L483P, A495P and Va1499=), RecTL (which includes RecNciI and p.D448H) and c.1263del+RecTL (which includes RecNciI, p.D448H and c.1263del) (FIGS. 2C1 and 2C2). The high homology and the frequent gene conversion between GBA and GBAP1 make Exon9-11 a very challenging region for standard secondary analysis pipelines. For example, since three positions in the GBAP1 reference sequence in hg38 erroneously contain the GBA bases (FIG. 2C1), GBA p.L483P reads would easily be aligned to GBAP1, causing false negative calls. In addition, in the population there exist GBAP1 haplotypes that have been partially converted to GBA and those converted bases would direct GBAP1 reads to align to GBA, causing false positive GBA variant calls at nearby positions (FIG. 2C1, purple shading/bottom two rows). Gauchian phased haplotypes throughout the homology region considering all reads that align to either GBA or GBAP1, and can thus call these variants accurately. This also allowed identification of bigger gene conversion events such as RecTL or c.1263del+RecTL, while standard pipelines would miss these as the variant reads align to GBAP1. Gauchian detected five samples carrying p.L483P, two samples carrying c.1263del and two samples carrying c.1263del+RecTL conversion, in addition to 42 samples carrying simple non-recombinant small variants in 1 kGP.

FIG. 2A1. Median mapping quality (red line) across 2504 1 kGP samples plotted for each position in the GBA/GBAP1 region (hg38). A median filter was applied in a 50 bp window. The eleven exons of GBA are shown as orange boxes. GBAP1 and MTX1 exons are shown as green and purple boxes, respectively. The 4 kb major homology region (98.1% sequence similarity, Exon9-11) between GBA and GBAP1 is shaded in pink in FIG. 2A1 (corresponding to the green boxed regions in FIG. 2A2) and highlights an area of low mapping accuracy. The light blue box shows the 10 kb unique region between the two genes in which copy number calling is performed in Gauchian. FIG. 2B1. Distribution of normalized depth in the 10 kb CN calling region in 2504 1 kGP samples, showing peaks at 1 (deletion), 2, and 3-8 (multiplications). This number plus two gave the total copies of both GBA and GBAP1 combined. The 10 kb unique region is a proxy for the 20.6 kb region between GBA and GBAP1 that would be lost or gained due to reciprocal recombination (breakpoints may vary). Normally a diploid sample would have 4 total copies of GBA+GBAP1 (2 copies each) and 2 copies of the 10 kb unique region. A deletion between GBA and GBAP1 would lead to one copy loss of the 10 kb region (now CN of 1) as well as one copy loss of GBA+GBAP1 (now CN of 3). Similarly, a duplication would lead to one copy gain of the 10 kb region (now CN3) as well as one copy gain of GBA+GBAP1 (now CN5). So CN(GBA+GBAP1) is two more than the CN of the 10 kb region.

FIG. 2B2 shows race-dependent distribution of CN. FIG. 2C1. Recombinant haplotypes in the Exon9-11 homology region, distinguished by GBA/GBAP1 differentiating bases (x axis). Reference genome sequences are shaded in yellow. There is an error on hg38 reference where the first three sites of GBAP1 show GBA bases, which could lead to alignment errors. The GBA recombinant haplotypes are shown in the white background, including those where one or a few nearby sites are mutated to the GBAP1 base, resulting from either gene conversion or fusion deletion. Gray bases indicate that the base can be either GBA or GBAP1 depending on the breakpoint position of the fusion/conversion. Shaded in purple are two example GBAP1 haplotypes that have been partially converted to GBA and cause false positive GBA variant calls. For the first example, particularly for hg38, where the first three sites are wrong for GBAP1 reference, the reverse-L483P variant on GBAP1 directs aligners to align GBAP1 reads to GBA, causing the nearby A495P FP call. For the second example, the reverse-c.1263del variant inserts 55 bp to GBAP1, driving GBAP1 reads to align to GBA, causing the nearby D448H FP call.

Detection of all Classes of GBA Variants is Possible with ONT Long-Read Sequencing

Cross-Validation

To seek validation of Gauchian, (Oxford Nanopore Technologies) ONT sequencing was performed for 14 samples where Gauchian detected a reciprocal recombinant (11 with copy number gains and 3 with copy number losses), two samples where Gauchian detected the non-reciprocal recombinant c.1263del+RecTL, 9 samples carrying SNVs (8 carrying p.L483P and 1 A456P) and 12 GBA-negative controls. These samples were selected from 1 kGP and AMP-PD and included 2 samples where GATK missed the p.L483P and 2 samples where GATK wrongly called the p.A456P variant. In all cases, ONT and Gauchian results were consistent, including in the cases where GATK results differ.

Additionally, since Gauchian had demonstrated evidence of multicopy CNVs, we used digital PCR to accurately quantify the copy number of the 20.6 kb region involved in recombination in 4 samples where Gauchian detected a copy number gain (copy number gained: 1, 3, 5 and 6, respectively). dPCR results were as expected, with the CN consistent with those detected by Gauchian and ONT (Table 2).

TABLE 2
Cross-validation data

| | Number of samples detected |
|---|---|
| Variants | by both Gauchian and ONT |
| Copy number gain (CN gained = 1-6) | 11* |
| Non-pathogenic fusion | 3 |
| Pathogenic fusion RecNciI | 1 (HG00422) |
| c.1263del + RecTL conversion | 2 |
| p.L483P | 8** |
| p.A495P | 1 |
| No GBA variants | 12*** |

*includes 4 samples where exact copy number has been confirmed by dPCR
**includes 2 samples where GATK did not detect p.L483P
***includes 2 samples where GATK wrongly called p.A495P

Prevalence of GBA Recombinant and Non-Recombinant Variants in Healthy, PD and LBD Populations

Having validated Gauchian, Gauchian was applied to Parkinson's disease (PD) and Lewy Body Dementia (LBD) cohorts from AMP-PD to provide the first large-scale analysis on GBA recombinant variants and estimate the prevalence of different GBA mutations in healthy, PD and LBD populations.

For CNVs with breakpoints that do not alter the GBA gene (copy number gains and non-pathogenic fusion alleles), there was no enrichment in PD nor in LBD cases versus controls (Table 3). Among Caucasians, CNVs were found in 18 (10 multiplications and 8 deletions) out of 2234 PD cases (0.81%) and 14 (7 multiplications and 7 deletions) out of 1214 controls (1.15%) (p-value=0.35, Fisher's exact test). Among Africans, CNVs (multiplications) were found in 2 out of 25 PD cases and 3 out of 33 controls (p-value=1). Among Caucasians, CNVs were found in 34 (21 multiplications and 13 deletions) out of 2598 LBD cases (1.31%) and 24 (11 multiplications and 13 deletions) out of 1941 controls (1.24%) (p-value=0.89). Across both 1 kGP and PD cohorts, CNVs (especially multiplications) were more than nine times more frequent overall in Africans than in Caucasians (1 kGP: 11.6% vs 0.6%. PD: 8.6% vs 0.9%). These results are consistent with the greater and still largely unexplored African genetic diversity, with recent evidence of African genomes demonstrating unexplored structural variation. Interestingly, 3 out of the 10 PD cases with multiplications have a second pathogenic GBA variant, and 4 out of the 21 LBD cases with multiplications have a second pathogenic GBA variant. Although multiplications do not alter the GBA gene themselves, multiplications could lead to a higher likelihood of acquiring a second GBA variant. This is consistent with what was found in RAPSODI and QSBB ONT data.

TABLE 3
Non-pathogenic CNVs in 1kGP, PD and LBD cohorts

| | 1kGP | | PD | | | | LBD | |
|---|---|---|---|---|---|---|---|---|
| | Caucasian | African | Caucasian | | African | | Caucasian | |
| | Control | Control | Case | Control | Case | Control | Case | Control |
| Deletion | 2 | 3 | 8 | 7 | 0 | 0 | 13 | 13 |
| Multiplication | 1 | 74 | 10* | 7 | 2 | 3 | 21** | 11 |
| Total | 503 | 661 | 2234 | 1214 | 25 | 33 | 2598 | 1941 |

| | | | | | |
|---|---|---|---|---|---|
| p-value | NA | NA | 0.35 | 1 | 0.89 |
| Fisher's exact test | | | | | |

*3 out of the 10 PD cases with multiplication have a second pathogenic GBA variant (L483P).
**4 out of the 21 LBD cases with multiplication have a second pathogenic GBA variant (L483P, D448H, c.1263del + RecTL). The fourth sample is a compound heterozygote for L483P and D448H.

In addition to benign CNVs, recombinant variants were detected in all three cohorts (Table 4). GBA recombinant variants are more common in LBD than PD (50/2598, 1.92% vs. 19/2325, 0.82%, p-value=0.0009). GATK variant calls were available for PD and LBD samples from AMP-PD. Due to sequence homology in Exon9-11, GATK undercalled all recombinant variants except D448H. For D448H, GATK called 2 false positives due to GBAP1 haplotypes with bases converted to GBA (see FIG. 2C). For all PD and LBD case+control populations, GATK called 35 recombinant variants and Gauchian called 77, more than doubling the variant calls.

Gauchian also detected simple non-recombinant variants in the three cohorts (Table 5). Again GBA variants are more common in LBD than PD.

TABLE 4
Recombinant variants in 1kGP, PD and LBD cohorts

| | | | | | RecNciI | | c.1263del + RecTL | | |
|---|---|---|---|---|---|---|---|---|---|
| | | L483P | D448H | c.1263del | Fusion | Conversion | Fusion | Conversion | Total |
| 1kGP | N = 2405 | 5 | 0 | 2 | 1 | 0 | 0 | 2 | 10 |
| PD | Case | 14 | 1 | 0 | 1 | 2 | 1 | 0 | 19 |
| | (N = 2325) | | | | | | | | |
| | Control | 6 | 1 | 0 | 0 | 0 | 0 | 0 | 7 |
| | (N = 1255) | | | | | | | | |
| LBD | Case | 23 | 4 | 6 | 10 | 3 | 2 | 2 | 50 |
| | (N = 2598) | | | | | | | | |
| | Control | 0 | 1 | 0 | 0 | 0 | 0 | 0 | 1 |
| | (N = 1941) | | | | | | | | |

| | | | | | | | | |
|---|---|---|---|---|---|---|---|---|
| PD + LBD | 43 | 7 | 6 | 11 | 5 | 3 | 2 | 77 |
| called by Gauchian | | | | | | | | |
| PD + LBD | 27 | 7 | 0 | 0 | 1 | 0 | 0 | 35 |
| called by GATK | | (+2 FP) | | | | | | |

TABLE 5
Non-recombinant variants in 1kGP, PD and LBD cohorts

| | | PD | | LBD | |
|---|---|---|---|---|---|
| | 1kGP | case | control | case | control |
| | N = 2405 | N = 2325 | N = 1255 | N = 2598 | N = 1941 |
| c.115 + 1G > A | 2 | 3 | 1 | 3 | 0 |
| c.259C > T(p.Arg87Trp) | 0 | 0 | 0 | 1 | 0 |
| c.354G > C(p.Lys118Asn) | 0 | 0 | 0 | 1 | 0 |
| c.475C > T(p.Arg159Trp) | 0 | 3 | 0 | 3 | 0 |
| c.508C > T(p.Arg170Cys) | 0 | 0 | 0 | 1 | 0 |
| c.667T > C(p.Trp223Arg) | 0 | 0 | 0 | 1 | 0 |
| c.680A > G(p.Asn227Ser) | 1 | 0 | 0 | 1 | 0 |
| c.703T > C(p.Ser235Pro) | 0 | 1 | 0 | 0 | 0 |
| c.721G > A(p.Gly241Arg) | 0 | 1 | 0 | 4 | 0 |
| c.754T > A(p.Phe252Ile) | 0 | 1 | 0 | 0 | 0 |
| c.764T > A(p.Phe255Tyr) | 0 | 1 | 0 | 0 | 0 |
| c.84dupG(p.Leu29Alafs*18) | 0 | 3 | 0 | 0 | 1 |
| c.887G > A(p.Arg296Gln) | 0 | 3 | 1 | 4 | 0 |
| c.894C > A(p.Phe298Leu) | 0 | 0 | 0 | 1 | 0 |
| c.914del(p.Pro305fs) | 0 | 0 | 0 | 1 | 0 |
| c.1043C > T(p.Ala348Val) | 0 | 0 | 0 | 1 | 0 |
| c.1049A > G(p.His350Arg) | 0 | 0 | 0 | 1 | 0 |
| c.1090G > A(p.Gly364Arg) | 0 | 0 | 0 | 1 | 0 |
| c.1093G > A(p.Glu365Lys) | 25 | 94 | 21 | 145 | 30 |
| c.l192C > T(p.Arg398Ter) | 0 | 0 | 0 | 1 | 0 |
| c.1223C > T(p.Thr408Met) | 9 | 40 | 10 | 65 | 32 |
| c.1226A > G(p.Asn409Ser) | 3 | 129 | 166 | 59 | 19 |
| c.1246G > A(p.Gly416Ser) | 0 | 0 | 0 | 1 | 0 |
| c.1259G > A(p.Trp420Ter) | 0 | 0 | 0 | 1 | 0 |
| c.1297G > T(p.Val433Leu) | 0 | 1 | 0 | 1 | 0 |
| c.1312G > A(p.Asp438Asn) | 0 | 0 | 0 | 1 | 0 |
| c.1448T > G(p.Leu483Arg) | 0 | 1 | 0 | 0 | 0 |
| c.1495G > C(p.Val499Leu) | 0 | 0 | 0 | 1 | 0 |
| c.1504C > T(p.Arg502Cys) | 2 | 2 | 0 | 8 | 0 |
| c.1603C > T(p.Arg535Cys) | 0 | 0 | 0 | 1 | 0 |
| c.1604G > A(p.Arg535His) | 0 | 6 | 3 | 4 | 1 |

### Gauchian—a WGS-Based GBA Caller

Gauchian, a WGS-based GBA caller disclosed herein uses a novel approach to overcome this challenge, building upon the strategies to solve closely related paralogs as described in the SMNIISMN2 caller (described in Chen et al. Spinal muscular atrophy diagnosis and carrier screening from genome sequencing data, Genet Med 22, 945-953 (2020), the content of which is incorporated herein by reference in its entirety) and the Cyrius CYP2D6 caller (described in Chen et al., Cyrius: accurate CYP2D6 genotyping using whole genome sequencing data, Pharmacogenomics J 21, 251-261 (2021), the content of which is incorporated herein by reference in its entirety). In some embodiments, the method of Gauchian can be applied to sequence reads from targeted sequencing, such as sequencing of 5, 10, 20, 30, 40, 50, 100, 200, or more genes.

First, Gauchian calculates the copy number of the 10 kb unique region (chr1:155220429-155230539, hg38) between GBA and GBAP1, following a similar targeted CNV calling method used by the SMN1/SMN2 caller and the Cyrius CYP2D6 caller. The number of reads aligned to this region is normalized and corrected for GC content and the copy number was called from a Gaussian mixture model. A deviation of this copy number (CN) from the expected two copies indicates the presence of a CNV. For example, one copy indicates a deletion and three copies indicate a duplication. Thus, this number plus two gives the total copies of both GBA and GBAP1 combined (compare FIG. 2B1 and FIG. 2B2). The total copies of both GBA and GBAP1 combined is abbreviated herein as CN(GBA+GBAP1).

Next Gauchian identifies the breakpoint of the CNV, following a similar approach as used by the Cyrius CYP2D6 caller. To do this, 82 reliable bases that differ between GBA and GBAP1 are used. Gauchian estimates the GBA CN at each of the 82 GBA/GBAP1 differentiating base positions based on CN(GBA+GBAP1) and the numbers of reads supporting GBA- and GBAP1-specific bases. CNV breakpoints are identified when the CN of GBA changes. For example, a switch from CN1 to CN2 indicates the breakpoint of a deletion and a switch from CN3 to CN2 indicates the breakpoint of a duplication. The exact breakpoint is further refined by haplotype phasing as described in the next paragraph.

To identify recombinant variants, Gauchian analyzes the 1.1 kb region (FIG. 2C) containing the critical GBA/GBAP1 recombinant variants (p.L483P, p.D448H, c.1263del, RecNciI, RecTL and c.1263del+RecTL). This region contains 10 GBA/GBAP1 base differences. Based on reads and read pairs, Gauchian phases all the haplotypes originating from either GBA or GBAP1 in this region and identifies hybrid haplotypes (i.e. a mixture of GBA and GBAP1 bases on the same haplotype). To assess the relative abundance of the different haplotypes, Gauchian uses CN(GBA+GBAP1) as well as haplotype-supporting read counts at the differentiating bases to call CN of each haplotype. Gauchian compares two scenarios: one copy of the wildtype GBA haplotype vs. two copies of the wildtype GBA haplotype. Gauchian determines which scenario is more likely given the number of supporting reads in the data. If Gauchian calls only one copy of the wildtype GBA haplotype, this indicates that the individual is a carrier of the disease-causing variant. If an individual is a carrier of more than one variant haplotype and there is no haplotype that carries the GBA base at all variant sites of interest, Gauchian calls this sample as compound heterozygous. Homozygous variants are called when the CN of the GBA base is called as 0. Finally, for simple small variants, Gauchian parses read alignments and calls the CN of variants as used by the SMN1/SMN2 caller and the Cyrius CYP2D6 caller.

CYP21A2 Variants

The RCCX module of the human MHC class III region includes a tandem repeat of about 30 kb with 99.6% similarity. The RCCX modules encodes RP1, C4A/B, CYP21A2, and TNXB. CYP21A1P is a pseudogene of CYP21A2. Gene recombinant variants of CYP21A2 can cause 21-Hydroxylase-Deficient Congenital Adrenal Hyperplasia (21-OHD CAH) with an incidence of 1:10,000-1:16,000 live births. C4A and C4B together form Complement Component 4 (C4). C4 deficiency is associated with autoimmune diseases such as lupus. Mutations in TNXB can cause Ehlers-Danlos syndrome.

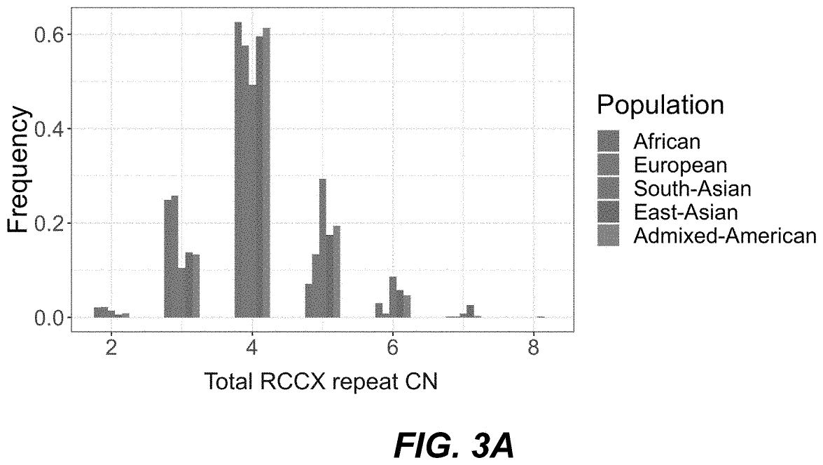

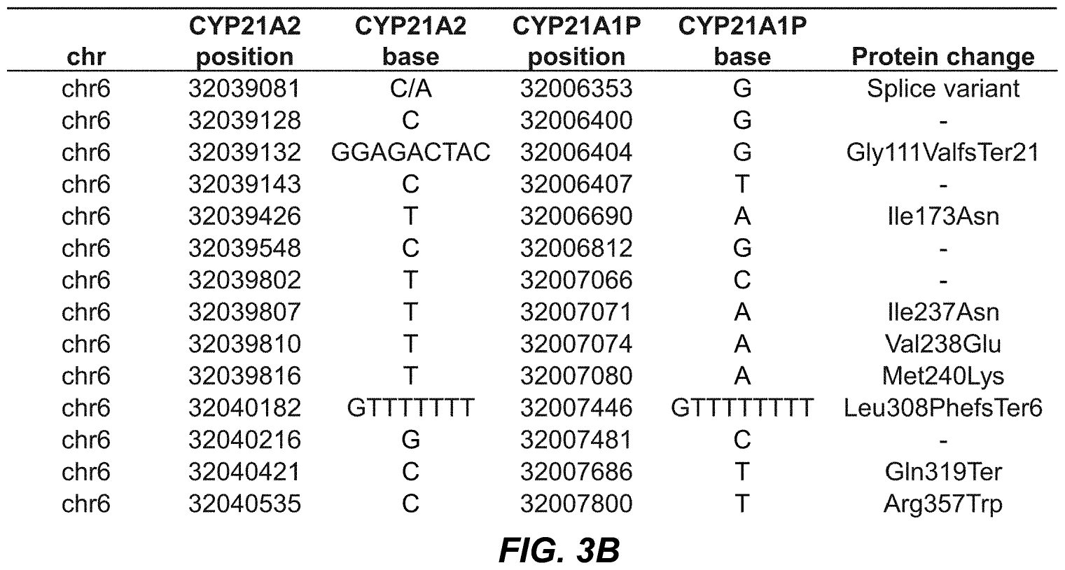

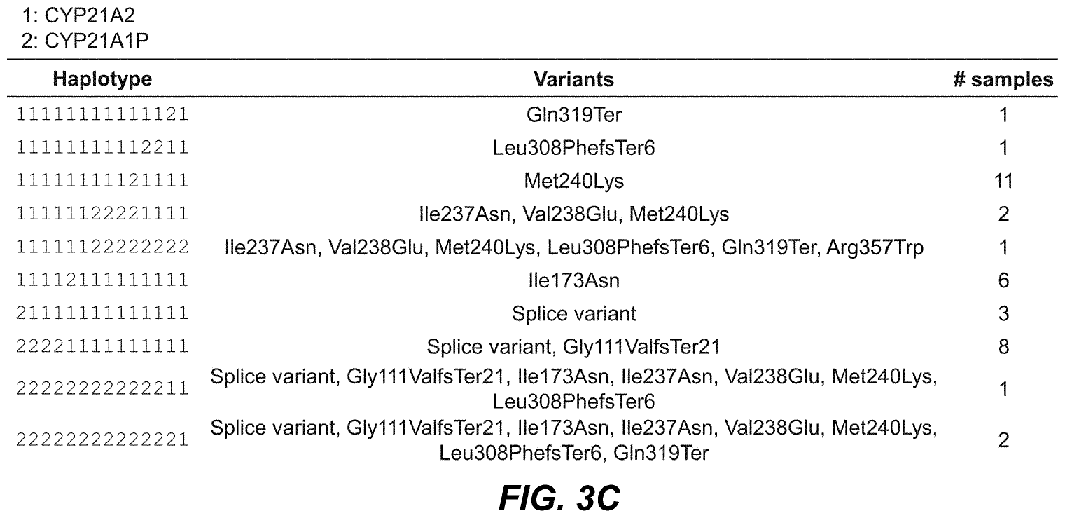

The RCCX repeats in samples of subjects were as described above. The CNs of the RCCX repeats in samples were determined using a Gaussian mixture model. FIG. 3A shows a race-dependent distribution of CN. Deletion breakpoints were identified by examining the switch in CN of differentiating SNP sites. C4A and C4B include five SNPs that mark the functional difference between C4A/C4B. Reads and read pairs aligned to fourteen differences between CYP21A2 and CYP21A1P, including nine gene recombinant variants, shown in FIG. 3B were analyzed with read backed phasing for haplotype phasing. Whether CYP21A2 wildtype haplotype was present at only one copy in a sample of a subject was tested based on depth/the number of supporting reads in the data. FIG. 3C shows a distribution of CYP21A2 haplotypes other than the CYP21A2 wildtype haplotype the subjects had. The CYP21A2 wildtype haplotype would be represented as 11111111111111 with all the bases being CYP21A2 bases at the 14 positions. The CYP21A2 haplotypes shown in FIG. 3C are not the CYP21A2 wildtype haplotype and thus would be represented by, for example, 11111111111121 indicating the 13th base of the is the CYP21A1P base while the remaining bases are CYP21A2 bases.

### Determining GBA Variants and Variant Status

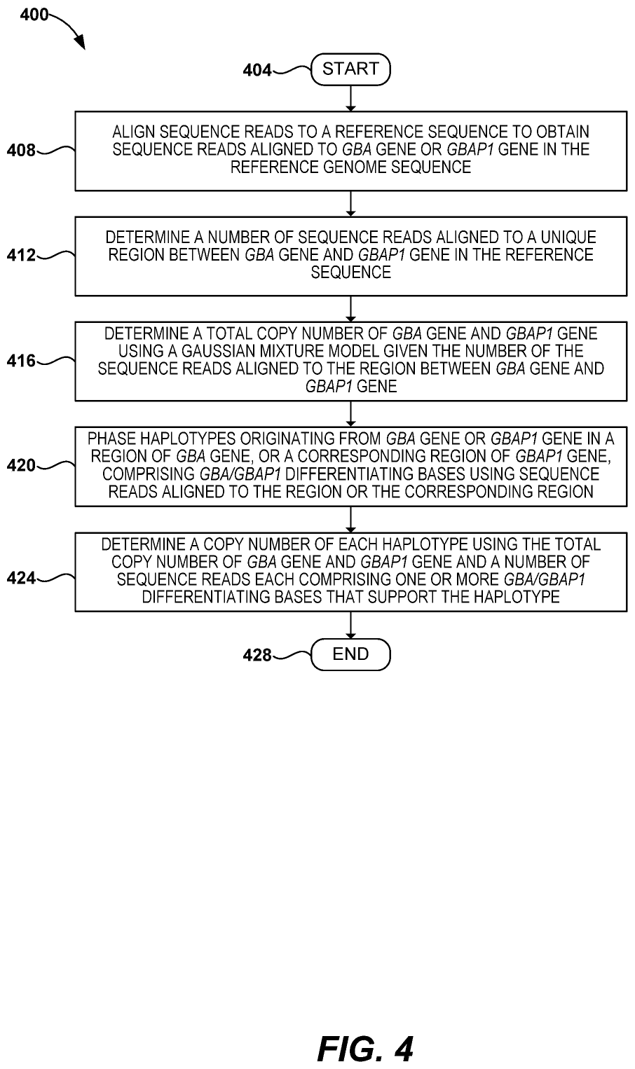

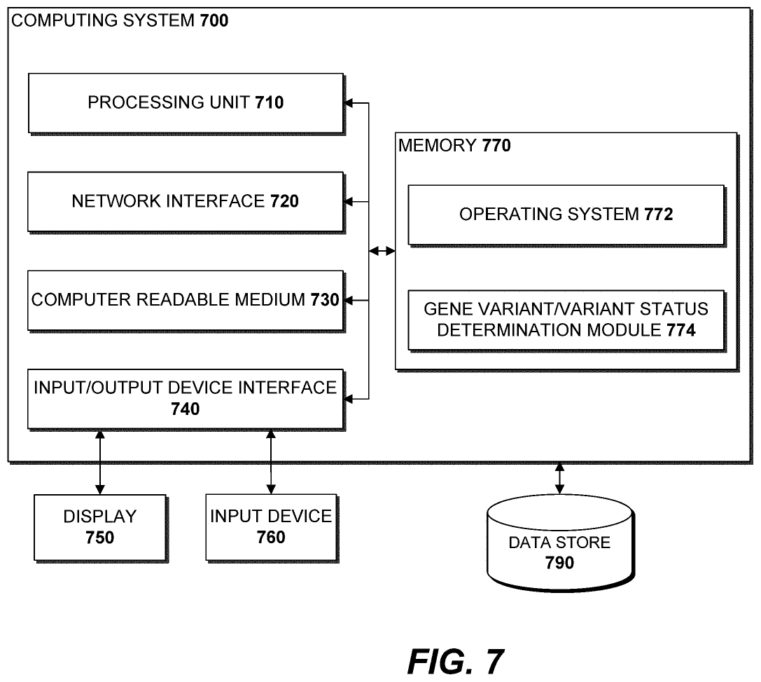

FIG. 4 is a flow diagram showing an exemplary method 400 of determining or identifying one or more GBA variant or GBA variant status. The method 400 may be embodied in a set of executable program instructions stored on a computer-readable medium, such as one or more disk drives, of a computing system. For example, the computing system 700 shown in FIG. 7 and described in greater detail below can execute a set of executable program instructions to implement the method 400. When the method 400 is initiated, the executable program instructions can be loaded into memory, such as RAM, and executed by one or more processors of the computing system 700. Although the method 400 is described with respect to the computing system 700 shown in FIG. 7, the description is illustrative only and is not intended to be limiting. In some embodiments, the method 400 or portions thereof may be performed serially or in parallel by multiple computing systems.

After the method 400 begins at block 404, the method 400 proceeds to block 408, where a computing system (e.g., the computing system 700 described with references to FIG. 7) can align a first plurality of sequence reads to a reference sequence (e.g., a reference genome sequence, such as hg19, or hg38) to obtain a second plurality of sequence reads aligned to GBA gene or GBAP1 gene in the reference sequence (including alignment of each of the second plurality of sequence reads to GBA gene or GBAP1 gene in the reference sequence). The computing system can receive the first plurality of sequence reads generated from a sample obtained from a subject. The computing system can store the first plurality of sequence reads in memory. The computing system can load the first plurality of sequence reads into memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, Calif.).

Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by whole genome sequencing (WGS). The WGS can be clinical WGS (cWGS). The sequence reads can comprise single-end sequence reads. The sequence reads can be generated by targeted sequencing, such as sequencing of 5, 10, 20, 30, 40, 50, 100, 200, or more genes. The sample can comprise cells, cell-free DNA, cell-free fetal DNA, amniotic fluid, a blood sample, a biopsy sample, or a combination thereof.

A sequence read can be aligned to GBA gene or GBAP1 gene in the reference sequence with an alignment quality score of zero or more. A sequence read can be aligned to GBA gene or GBAP1 gene in the reference sequence with an alignment quality score of about zero (e.g., when a sequence is aligned to a region where the gene and the gene paralog are highly homologous). The computing system can align sequence reads to the reference sequence using an aligner or an alignment method such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The method 400 proceeds from block 408 to block 412, where the computing system determines a number (e.g., a normalized and/or corrected number) of the sequence reads of the second plurality of sequence reads aligned to a unique region between GBA gene and GBAP1 gene in the reference sequence. The unique region between GBA gene and GBAP1 gene in the reference sequence can comprise a unique region about 10 kilobases in length. The unique region between GBA gene and GBAP1 gene in the reference sequence can comprise chr1:155220429-155230539 of hg38 or a corresponding region of a reference human genome sequence.

The computing system can determine a normalized number of the sequence reads aligned to the unique region between GBA gene and GBAP1 gene in the reference sequence. The computing system can determine the normalized number of the sequence reads aligned to the unique region between GBA gene and GBAP1 gene in the reference sequence using (1a) a depth of the sequence reads aligned to the unique region between the GBA gene and GBAP1 gene, (1b) a length of the unique region, (2a) a depth of sequence reads of the first plurality of sequence reads aligned to each of a plurality of regions of the reference sequence other than a genetic locus comprising GBA gene and GBAP1 gene, and/or (2b) a length of each of the plurality of regions of the reference other than the genetic locus comprising GBA gene and GBAP1 gene.

The computing system can determine a normalized, corrected number of the sequence reads aligned to the unique region between GBA gene and GBAP1 gene in the reference sequence from the normalized number of the sequence reads aligned to the unique region between GBA gene and GBAP1 gene in the reference sequence. To determine the normalized, corrected number of the sequence reads aligned to the unique region between GBA gene and GBAP1 gene in the reference sequence, the computing system can determine a normalized, GC content-corrected number of the sequence reads aligned to the unique region between GBA gene and GBAP1 gene in the reference sequence from the normalized number of the sequence reads aligned to the unique region between GBA gene and GBAP1 gene in the reference sequence. The computing system can determine the normalized, GC content-corrected number of the sequence reads aligned to the unique region between GBA gene and GBAP1 gene in the reference sequence from the normalized number of the sequence reads aligned to the unique region between GBA gene and GBAP1 gene in the reference sequence using (1) a GC content of the unique region between the GBA gene and GBAP1. The computing system can determine the normalized, GC content-corrected number of the sequence reads aligned to the unique region between GBA gene and GBAP1 gene in the reference sequence from the normalized number of the sequence reads aligned to the unique region between GBA gene and GBAP1 gene in the reference sequence using (1) a GC content of the unique region between the GBA gene and GBAP1 gene and/or (2) a GC content of each of one or more regions of the reference sequence other than a genetic locus comprising GBA gene and GBAP1 gene (or one or more regions of the reference sequence not comprising GBA gene and GBAP1 gene). For example, the computer system can determine the normalized, GC content-corrected number of the sequence reads aligned to the unique region between GBA gene and GBAP1 gene in the reference sequence using (1) the GC content of the unique region between the GBA gene and GBAP1 gene and (2) the GC content of a region of the reference sequence other than the genetic locus comprising GBA gene and GBAP1 gene. As another example, the computer system can determine the normalized, GC content-corrected number of the sequence reads aligned to the unique region between GBA gene and GBAP1 gene in the reference sequence using (1) the GC content of the unique region between the GBA gene and GBAP1 gene and (2) the GC contents of multiple regions (e.g., 2, 3, 4, 5, 10, 20, 30, 40, 50, 100, 200, 300, 400, 500, 1000, 2000, 3000, 4000, 5000, 10000, or more regions) of the reference sequence other than the genetic locus comprising GBA gene and GBAP1 gene.

The method 400 proceeds from block 412 to block 416, where the computing system determines a total copy number of GBA gene and GBAP1 gene using a Gaussian mixture model comprising a plurality of Gaussians each representing a different integer copy number, given the number of the sequence reads (e.g., normalized and/or corrected sequence reads) aligned to the region between GBA gene and GBAP1 gene. The computing system can determine the total copy number of GBA gene and GBAP1 gene using the Gaussian mixture model, given the normalized number of the sequence reads aligned to the region between GBA gene and GBAP1 gene. The computing system can determine the total copy number of GBA gene and GBAP1 gene using the Gaussian mixture model, given the normalized, corrected number of the sequence reads aligned to the region between GBA gene and GBAP1 gene.

The total copy number can be, for example, 2, 3, 4, 5, 6, 7, 8, 9, 10, or more. The Gaussian mixture model can comprise a one-dimensional Gaussian mixture model. The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers 0 to 10. A mean of each of the plurality of Gaussians can be the integer copy number represented by the Gaussian. A mean of each of the plurality of Gaussians can be the integer copy number represented by the Gaussian (e.g., copy numbers of 1, 2, 3, 4, 5, 6, 7, 8, 9, 10, 11, 12, 13, 14, 15, or more). The standard deviation of a Gaussian can be or be about, for example, 0.1, 0.2, 0.3, 0.4, 0.5, 0.6, 0.7, 0.8, 0.9, 1, or more. The plurality of Gaussians of the Gaussian mixture model can comprise, for example, 5, 6, 7, 8, 9, 10, 11, 12, 13, 14, 15, 16, or more, Gaussians. For example, the plurality of Gaussians of the Gaussian mixture model can comprise 5 Gaussians.

To determine the total copy number of GBA gene and GBAP1 gene, the computing system can determine a copy number of the region between GBA gene and GBAP1 gene using the Gaussian mixture model, given the normalized number of the sequence reads aligned to the region between GBA gene and GBAP1 gene. The total copy number of GBA gene and GBAP1 gene can be the copy number of the region between GBA gene and GBAP1 gene plus two.

The computing system can determine the total copy number of GBA gene and GBAP1 gene using a Gaussian mixture model and a predetermined posterior probability threshold, given the normalized number of the sequence reads aligned to the region between GBA gene and GBAP1 gene. The predetermined posterior probability threshold can be or be about, for example, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or more. For example, the predetermined posterior probability threshold is 0.95.

The method 400 proceeds from block 416 to block 420, where the computing system phases one or more haplotypes originating from GBA gene or GBAP1 gene in a region of GBA gene, or a corresponding region of GBAP1 gene, comprising a plurality of GBA/GBAP1 differentiating bases (or positions or sites of differentiating bases) using sequence reads of the second plurality of sequence reads aligned to the region, or the corresponding region, comprising the plurality of GBA/GBAP1 differentiating bases. For example, a sequence read can be aligned to the reference sequence such that the sequence read overlaps a GBA/GBAP1 differentiating base (or the site of the GBA/GBAP1 differentiating base) or a base of the sequence read is aligned to the GBA/GBAP1 paralog differentiating base (or the site of the GBA/GBAP1 paralog differentiating base). A sequence read of the second plurality of sequence reads can be aligned to the region of GBA gene, or the corresponding region of GBAP1 gene, comprising the plurality of GBA/GBAP1 differentiating bases with an alignment quality score of zero or more.

The one or more haplotypes comprises a wildtype GBA haplotype, a wildtype GBAP1 haplotype, and/or a GBA/GBAP1 hybrid haplotype. A GBA/GBAP1 hybrid haplotype can include both GBA bases and GBAP1 bases. A GBA/GBAP1 hybrid haplotype can be a recombinant variant. The GBA/GBAP1 hybrid haplotype can comprise a GBA variant haplotype or a GBAP1 variant haplotype. A haplotype can comprise a reciprocal recombinant variant. A haplotype can comprise a non-reciprocal recombinant variant or a gene conversion variant. The reference sequence can comprise a reference genome sequence.

To phase the one or more haplotypes originating from GBA gene or GBAP1 gene, the computing system can analyze linkage information between GBA/GBAP1 differentiating bases of the plurality of GBA/GBAP1 differentiating bases using sequence reads of the second plurality of sequence reads aligned to the region, or the corresponding region, comprising the plurality of GBA/GBAP1 differentiating bases. The computing system can phase the one or more haplotypes originating from GBA gene or GBAP1 gene using sequence reads of the second plurality of sequence reads each aligned two or more of the plurality of GBA/GBAP1 differentiating bases. For example, referring to FIG. 1, assuming gene A and gene B shown in the figure are GBA gene or GBAP1 gene, respectively, one read pair that covers Site 1 and Site 4 of differentiating bases can indicate the haplotype (haplotype x) the read pair originates from has GBA gene base at Site 1 and GBA gene base at Site 4. One read pair that covers Site 3 and Site 5 of differentiating bases can indicate the haplotype (haplotype y) the read pair originates from has GBA gene base at Site 3 and GBAP1 gene base at Site 5. One read that covers Site 4 and Site 5 of differentiating bases can indicate the haplotype (haplotype y) the read originates from has GBA gene base at Site 4 and GBAP1 gene base at Site 5. The computing system can phase all the haplotypes originating from either GBA gene or GBAP1 gene in the region with the set of five reliable base differences and identify haplotypes of GBA gene (haplotype 1) and GBAP1 gene (haplotype 2) and hybrid haplotypes (haplotypes 3 and 4). The number of haplotypes, the bases of the haplotypes at the sites, the number of sites, and the sites (e.g., Sites 1 and 4, or Sites 3 and 5) sequence reads cover are shown in FIG. 1 for illustration only and are not intended to be limiting.

The region of GBA gene, or the corresponding region of GBAP1 gene, comprising the plurality of GBA/GBAP1 differentiating bases can be about 1.1 (or 0.8, 0.9, 1, 1.2, 1.3, or more) kilobases in length. The region of GBA gene, or the corresponding region of GBAP1 gene, comprising the plurality of GBA/GBAP1 differentiating bases can comprise exons 9-11 of GBA gene, or GBAP1 gene, respectively. The region of GBA gene, or the corresponding region of GBAP1 gene, comprising the plurality of GBA/GBAP1 differentiating bases can comprise p.L483P, p.D448H, c.1263del, RecNciI, RecTL, and c.1263del+RecTL. The plurality of GBA/GBAP1 differentiating bases can comprise 10 GBA/GBAP1 differentiating bases.

The method 400 proceeds from block 420 to block 424, where the computing system determines a copy number of each of the one or more haplotypes using the total copy number of GBA gene and GBAP1 gene and a number of sequence reads of the second plurality of sequence reads each comprising one or more of the plurality of GBA/GBAP1 differentiating bases that support the haplotype. The copy number of a haplotype can be, for example, 1, 2, 3, 4 or more.

The computing system can determine a GBA status (e.g., carrier, compound heterozygous, or homozygous) of the subject using the one or more haplotypes originating from GBA gene or GBAP1 gene in the region of GBA gene, or the corresponding region of GBAP1 gene, and/or the copy number of each of the one or more haplotypes. The computing system can generate a user interface (UI), such as a graphical user interface, comprising a UI element representing or comprising the GBA status. The UI can comprise the GBA status as a part of a UI element. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

Carrier. To determine the copy number of each of the one or more haplotypes, the computing system can determine a likelihood of one copy of a wildtype GBA haplotype is higher than a likelihood of two copies of the wildtype GBA haplotype given the number of sequence reads of the second plurality of sequence reads each comprising one or more of the plurality of GBA/GBAP1 differentiating bases that support the wildtype GBA haplotype. The computing system can determine the likelihood of one copy of the wildtype GBA haplotype is higher than the likelihood of two copies of the wildtype GBA haplotype for (e.g., using the sequence reads of) one, one or more (e.g., two, three, or four), or each of the one or more haplotypes. The likelihood difference can be, for example, 1%, 2%, 3%, 5%, 10%, 15%, 20%, or more. The computing system can determine the copy number of the wildtype GBA haplotype is one. The computing system can determine the likelihood of one copy of the wildtype GBA haplotype is higher than the likelihood of two copies of the wildtype GBA haplotype for each of one or more pairs (or all pairs) of consecutive GBA/GBAP1 differentiating bases of the plurality of GBA/GBAP1 differentiating bases for which a first haplotype of the one or more haplotypes comprises GBA bases at the consecutive GBA/GBAP1 differentiating bases and a second haplotype of the one or more haplotypes comprises a GBA base and a GBAP1 base (or a GBAP1 base and a GBA base) at the consecutive GBA/GBAP1 differentiating bases. The first haplotype can comprise the GBA bases at the consecutive GBA/GBAP1 differentiating bases. The second haplotype can comprise a transition from the GBA base to the GBAP1 base (or from the GBAP1 base to the GBA base) between the consecutive GBA/GBAP1 differentiating bases. Consecutive GBA/GBAP1 differentiating bases are consecutive within the plurality of GBA/GBAP1 differentiating bases, whether the consecutive GBA/GBAP1 differentiating bases are adjacent bases in the reference sequence. For example, GBA/GBAP1 differentiating bases at Site 5 and Site 6 (or positions of differentiating bases) in the examples below are consecutive GBA/GBAP1 differentiating bases, whether the consecutive GBA/GBAP1 differentiating bases are adjacent bases in the reference sequence. The computing system can combine (e.g., averaging or weighted averaging) the likelihoods determined for each of one or more pairs (or all pairs) of consecutive GBA/GBAP1 differentiating bases to determine the likelihood of one copy of the wildtype GBA haplotype is higher than the likelihood of two copies of the wildtype GBA haplotype.

The computing system can determine the likelihood of one copy of the wildtype GBA haplotype is higher than the likelihood of two copies of the wildtype GBA haplotype for each of one or more pairs (or all pairs) of consecutive GBA/GBAP1 differentiating bases given (1) a number of sequence reads of the second plurality of sequence reads each comprising the GBA bases at the consecutive GBA/GBAP1 differentiating bases. The computing system can determine the likelihood of one copy of the wildtype GBA haplotype is higher than the likelihood of two copies of the wildtype GBA haplotype for each of one or more pairs (or all pairs) of consecutive GBA/GBAP1 differentiating bases given (2) a number of sequence reads of the second plurality of sequence reads each comprising the GBA base and the GBAP1 base (or the GBAP1 base and the GBA base) at the consecutive GBA/GBAP1 differentiating bases, and/or (3) a number of sequence reads of the second plurality of sequence reads each comprising the GBAP1 base and the GBA base at the consecutive GBA/GBAP1 differentiating bases. In some embodiments, the computing system can determine the likelihood of one copy of the wildtype GBA haplotype is higher than the likelihood of two copies of the wildtype GBA haplotype for each of one or more pairs (or all pairs) of consecutive GBA/GBAP1 differentiating bases given (4) a number of sequence reads of the second plurality of sequence reads each comprising the GBAP1 bases at the consecutive GBA/GBAP1 differentiating bases.

For example, by analyzing linkage information between GBA/GBAP1 differentiating bases, the following haplotypes (at six bases or sites or positions of differentiating bases for illustrative purposes only) can be determined for a subject:

| | Site/Differentiating Base | | | | | |
|---|---|---|---|---|---|---|
| Haplotype | 1 | 2 | 3 | 4 | 5 | 6 |
| 1 | GBA | GBA | GBA | GBA | GBA | GBA |
| 2 | GBA | GBA | GBA | GBA | GBA | GBAP1 |
| 3 | GBAP1 | GBAP1 | GBAP1 | GBAP1 | GBAP1 | GBAP1 |

A transition from GBA gene base to GBAP1 gene base occurs between Site 5 and Site 6 for haplotype 2. For example, the number of reads with the GBA bases at Site 5 and Site 6 is 98, the number of reads with the GBA base at Site 5 and the GBAP1 base at Site 6 is 105, and the number of reads with the GBAP1 bases at Site 5 and 6 is 190. The computing system can determine the likelihood of one copy of the wildtype GBA haplotype is higher than the likelihood of two copies of the wildtype GBA haplotype given the number of reads with the GBA bases at Site 5 and 6 is 98, the number of reads with the GBA base at Site 5 and the GBAP1 base at Site 6 is 105. The computing system can determine the likelihood of one copy of the wildtype GBA haplotype is higher than the likelihood of two copies of the wildtype GBA haplotype without using the reads from the wildtype GBAP1 haplotype.

As another example, by analyzing linkage information between GBA/GBAP1 differentiating bases, the following haplotypes (at six sites for illustrative purposes only) can be determined for a subject:

| | Site/Differentiating Base | | | | | |
|---|---|---|---|---|---|---|
| Haplotype | 1 | 2 | 3 | 4 | 5 | 6 |
| 1 | GBA | GBA | GBA | GBA | GBA | GBA |
| 2 | GBA | GBA | GBA | GBA | GBA | GBAP1 |
| 3 | GBAP1 | GBAP1 | GBAP1 | GBAP1 | GBAP1 | GBA |
| 4 | GBAP1 | GBAP1 | GBAP1 | GBAP1 | GBAP1 | GBAP1 |

A transition from the GBA base to the GBAP1 base occurs between Site 5 and Site 6 for haplotype 2. For example, the number of reads with the GBA bases at Site 5 and 6 is 98, the number of reads with the GBA base at Site 5 and the GBAP1 base at Site 6 is 105, the number of reads with the GBAP1 base at Site 5 and the GBA base at Site 6 is 95, and the number of reads with the GBAP1 bases at Site 5 and Site 6 is 104. The computing system can determine the likelihood of one copy of the wildtype GBA haplotype is higher than the likelihood of two copies of the wildtype GBA haplotype given the number of reads with the GBA bases at Site 5 and 6 is 98, the number of reads with the GBA base at Site 5 and the GBAP1 base at Site 6 is 105. The computing system can determine the likelihood of one copy of the wildtype GBA haplotype is higher than the likelihood of two copies of the wildtype GBA haplotype without using the reads from the wildtype GBAP1 haplotype. The computing system can determine the likelihood of one copy of the wildtype GBA haplotype is higher than the likelihood of two copies of the wildtype GBA haplotype without using the reads with the GBAP1 base at Site 5 and the GBA base at Site 6 because that haplotype (haplotype 3) has mostly GBAP1 bases at the sites/positions of differentiating bases and thus is unlikely a GBA variant haplotype/is likely to be a GBAP1 variant haplotype.

The copy number of the wildtype GBA haplotype can be one (i.e., a carrier of a GBA variant haplotype). The computing system can determine the GBA status of the subject as a carrier of a GBA variant haplotype. The one or more haplotypes can comprise four haplotypes. The total copy number of GBA gene and GBAP1 gene can be four. The copy number of each of the four haplotypes can be one (e.g., one copy of wildtype GBA haplotype, one copy of a GBA variant haplotype, one copy of GBAP1 wildtype haplotype, and one copy of a haplotype with GBAP1 bases at a high percentage (such as 80%, 85%, 90%, 95%, or more) of the differentiating bases and thus is unlikely a GBA variant haplotype/is likely a GBAP1 variant haplotype. The computing system can determine the GBA status of the subject as a carrier of a GBA variant haplotype. For example, by analyzing linkage information between GBA/GBAP1 differentiating bases, the following haplotypes (at six sites for illustrative purposes only) can be determined for a subject:

| | No. of | Site/Differentiating Base | | | | | |
|---|---|---|---|---|---|---|---|
| Haplotype | Copy(ies) | 1 | 2 | 3 | 4 | 5 | 6 |
| 1 | 1 | GBA | GBA | GBA | GBA | GBA | GBA |
| 2 | 1 | GBA | GBA | GBAP1 | GBAP1 | GBA | GBA |
| 3 | 1 | GBAP1 | GBAP1 | GBAP1 | GBA | GBAP1 | GBAP1 |
| 4 | 1 | GBAP1 | GBAP1 | GBAP1 | GBAP1 | GBAP1 | GBAP1 |

Because the subject has one copy of wildtype GBA haplotype and one copy of a GBA variant haplotype (and one copy of the wildtype GBAP1 haplotype, and one copy of a haplotype with mostly GBAP1 bases at the sites/positions of differentiating bases and thus is unlikely a GBA variant haplotype/is likely a GBAP1 variant haplotype), the subject is a carrier of a GBA variant haplotype.

The one or more haplotypes can comprise three haplotypes. The total copy number of GBA gene and GBAP1 gene can be four. The copy number of the wildtype GBA haplotype, the GBA variant haplotype, and the wildtype GBAP1 haplotype (or a haplotype with GBAP1 bases at a high percentage of the differentiating bases, such as 80%, 85%, 90%, 95%, or more) can be one, one, and two, respectively. The computing system can determine the GBA status of the subject as a carrier of a GBA variant haplotype. For example, by analyzing linkage information between GBA/GBAP1 differentiating bases, the following haplotypes (at six sites for illustrative purposes only) can be determined for a subject:

| | No. of | Site/Differentiating Base | | | | | |
|---|---|---|---|---|---|---|---|
| Haplotype | Copy(ies) | 1 | 2 | 3 | 4 | 5 | 6 |
| 1 | 1 | GBA | GBA | GBA | GBA | GBA | GBA |
| 2 | 1 | GBA | GBA | GBAP1 | GBAP1 | GBA | GBA |
| 3 | 2 | GBAP1 | GBAP1 | GBAP1 | GBAP1 | GBAP1 | GBAP1 |

Because the subject has one copy of wildtype GBA haplotype and one copy of a GBA variant haplotype, the subject is a carrier of a GBA variant haplotype.

Compound heterozygous. The one or more haplotypes can comprise two or more GBA variant haplotypes. None of the two or more GBA variant haplotypes can comprise a GBA base at each of the plurality of GBA/GBAP1 differentiating bases. None of the two or more GBA variant haplotypes can comprise GBA bases at all of the plurality of GBA/GBAP1 differentiating bases. Each of the two or more GBA variant haplotypes can comprise one or more GBAP1 bases at one or more of the plurality of GBA/GBAP1 differentiating bases. The computing system can determine the GBA status of the subject as compound heterozygous of GBA variant haplotypes. For example, by analyzing linkage information between GBA/GBAP1 differentiating bases, the following haplotypes (at six sites for illustrative purposes only) can be determined for a subject:

| | No. of | Site/Differentiating Base | | | | | |
|---|---|---|---|---|---|---|---|
| Haplotype | Copy(ies) | 1 | 2 | 3 | 4 | 5 | 6 |
| 1 | 1 | GBAP1 | GBA | GBA | GBA | GBA | GBA |
| 2 | 1 | GBA | GBA | GBAP1 | GBAP1 | GBA | GBA |
| 3 | 2 | GBAP1 | GBAP1 | GBAP1 | GBAP1 | GBAP1 | GBAP1 |

Because the subject does not have any copy of the wildtype GBA haplotype and has one copy of each of two GBA variant haplotypes, the subject is compound heterozygous of GBA variant haplotypes.

Homozygous. The one or more haplotypes can comprise an identical base (e.g., a GBA base or a GBAP1 base) at a GBA/GBAP1 differentiating base or at each of two or more of the plurality of GBA/GBAP1 differentiating bases. The computing system can determine the subject is homozygous (e.g., homozygous for the wildtype GBAP1 gene haplotype or homozygous for a GBA variant haplotype) at one or more of the plurality of the GBA/GBAP1 differentiating bases.

The computing system can determine a copy number of a GBA base at each of one or more of the plurality of GBA/GBAP1 differentiating bases is zero using sequence reads of the second plurality of sequence reads each comprising a base at the GBA/GBAP1 differentiating base that is not the GBA base. The base at the GBA/GBAP1 differentiating base that is not the GBA base can be a GBAP1 base. The computing system can determine the GBA status of the subject is homozygous for the GBA variant haplotype at one, one or more, or each of the one or more of the plurality of GBA/GBAP1 differentiating bases. For example, based on the plurality of GBA/GBAP1 differentiating bases, the computing system can determine the copy number (CN) of a GBA base. Using the number of reads supporting a GBA base or a GBAP1 base, as well as the total CN of the GBA gene and the GBAP1 gene, the most likely combination of the CN of the GBA base and the CN of the GBAP1 base can be determined. If the CN of the GBA base is determined as 0, this indicates that the subject has no copy of the wildtype GBA gene haplotype (the haplotype that carries the GBA base at a variant site of interest), and is homozygous for the GBA gene variant haplotype.

The method 400 ends at block 428.

### Determining CYP21A2 Variants and Variant Status

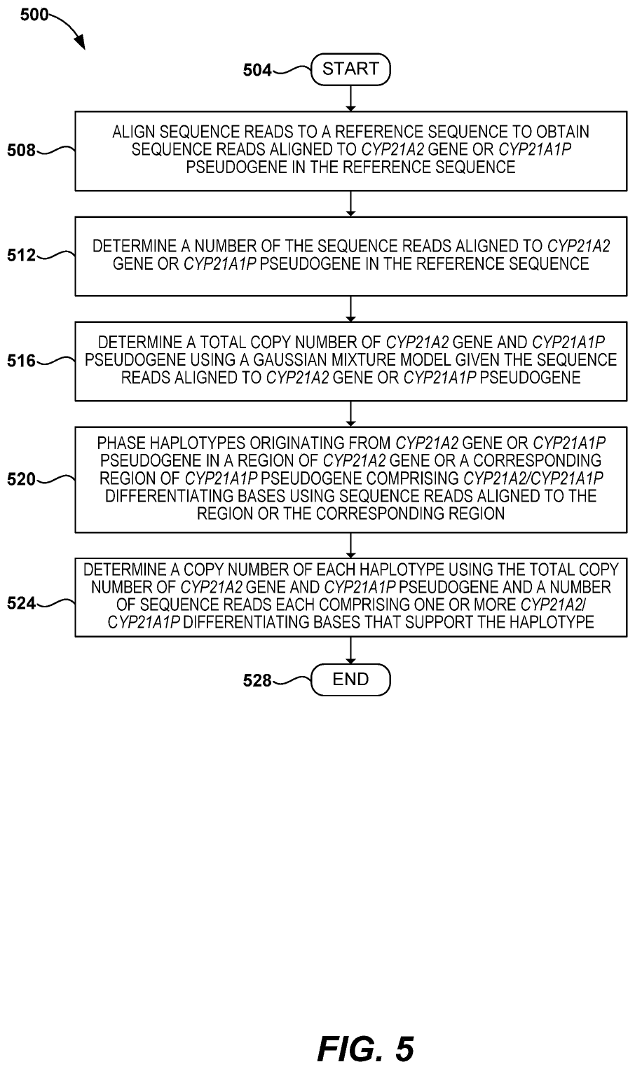

FIG. 5 is a flow diagram showing an exemplary method 500 of determining or identifying one or more CYP21A2 variants or variant status. The method 500 may be embodied in a set of executable program instructions stored on a computer-readable medium, such as one or more disk drives, of a computing system. For example, the computing system 700 shown in FIG. 7 and described in greater detail below can execute a set of executable program instructions to implement the method 500. When the method 500 is initiated, the executable program instructions can be loaded into memory, such as RAM, and executed by one or more processors of the computing system 700. Although the method 500 is described with respect to the computing system 700 shown in FIG. 7, the description is illustrative only and is not intended to be limiting. In some embodiments, the method 500 or portions thereof may be performed serially or in parallel by multiple computing systems.

After the method 500 begins at block 504, the method 500 proceeds to block 508, where a computing system (e.g., the computing system 700 described with references to FIG. 7) aligns a first plurality of sequence reads to a reference sequence (e.g., a reference genome sequence, such as hg19 or hg38) to obtain a second plurality of sequence reads aligned to CYP21A2 gene or CYP21A1P pseudogene in the reference sequence (including alignment of each of the second plurality of sequence reads to CYP21A2 gene or CYP21A1P gene in the reference sequence). The computing system can receive the first plurality of sequence reads generated from a sample obtained from a subject. The computing system can store the first plurality of sequence reads in memory. The computing system can load the first plurality of sequence reads into memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, Calif.).

Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by whole genome sequencing (WGS). The WGS can be clinical WGS (cWGS). The sample can comprise cells, cell-free DNA, cell-free fetal DNA, amniotic fluid, a blood sample, a biopsy sample, or a combination thereof.

A sequence read can be aligned to CYP21A2 gene or CYP21A1P pseudogene in the reference sequence with an alignment quality score of zero or more. A sequence read can be aligned to CYP21A2 gene or CYP21A1P pseudogene in the reference sequence with an alignment quality score of about zero (e.g., when a sequence is aligned to a region where the gene and the gene paralog are highly homologous). The computing system can align sequence reads to the reference sequence using an aligner or an alignment method such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The method 500 proceeds from block 508 to block 512, where the computing system determines a number (e.g., a normalized and/or corrected number) of the sequence reads of the second plurality of sequence reads aligned to CYP21A2 gene or CYP21A1P pseudogene in the reference sequence.

The computing system can determine a normalized number of the sequence reads aligned to CYP21A2 gene or CYP21A1P pseudogene in the reference sequence. The computing system can determine the normalized number of the sequence reads aligned to CYP21A2 gene or CYP21A1P pseudogene in the reference sequence using (1a) a depth of the sequence reads aligned to CYP21A2 gene or CYP21A1P pseudogene, (1b) a length of the unique region, (2a) a depth of sequence reads of the first plurality of sequence reads aligned to each of a plurality of regions of the reference sequence other than a genetic locus comprising CYP21A2 gene and CYP21A1P pseudogene, and/or (2b) a length of each of the plurality of regions of the reference other than the genetic locus comprising CYP21A2 gene and CYP21A1P pseudogene.

The computing system can determine a normalized, corrected number of the sequence reads aligned to CYP21A2 gene or CYP21A1P pseudogene in the reference sequence from the normalized number of the sequence reads aligned to CYP21A2 gene or CYP21A1P pseudogene in the reference sequence. To determine the normalized, corrected number of the sequence reads aligned to CYP21A2 gene or CYP21A1P pseudogene in the reference sequence, the computing system can determine a normalized, GC content-corrected number of the sequence reads aligned to CYP21A2 gene or CYP21A1P pseudogene in the reference sequence from the normalized number of the sequence reads aligned to CYP21A2 gene or CYP21A1P pseudogene in the reference sequence. The computing system can determine the normalized, GC content-corrected number of the sequence reads aligned to CYP21A2 gene or CYP21A1P pseudogene in the reference sequence from the normalized number of the sequence reads aligned to CYP21A2 gene or CYP21A1P pseudogene in the reference sequence using (1) a GC content of CYP21A2 gene or CYP21A1P pseudogene. The computing system can determine the normalized, GC content-corrected number of the sequence reads aligned to CYP21A2 gene or CYP21A1P pseudogene in the reference sequence from the normalized number of the sequence reads aligned to CYP21A2 gene or CYP21A1P pseudogene in the reference sequence using (1) a GC content of CYP21A2 gene or CYP21A1P pseudogene and/or (2) a GC content of each of one or more regions of the reference sequence other than a genetic locus comprising CYP21A2 gene and CYP21A1P pseudogene (or one or more regions of the reference sequence not comprising CYP21A2 gene and CYP21A1P pseudogene). For example, the computing system can determine the normalized, GC content-corrected number of the sequence reads aligned to CYP21A2 gene or CYP21A1P pseudogene in the reference sequence from the normalized number of the sequence reads aligned to CYP21A2 gene or CYP21A1P pseudogene in the reference sequence using (1) the GC content of CYP21A2 gene or CYP21A1P pseudogene and (2) the GC content of a region of the reference sequence other than the genetic locus comprising CYP21A2 gene and CYP21A1P pseudogene. As another example, the computing system can determine the normalized, GC content-corrected number of the sequence reads aligned to CYP21A2 gene or CYP21A1P pseudogene in the reference sequence from the normalized number of the sequence reads aligned to CYP21A2 gene or CYP21A1P pseudogene in the reference sequence using (1) the GC content of CYP21A2 gene or CYP21A1P pseudogene and (2) the GC content of multiple regions (e.g., 2, 3, 4, 5, 10, 20, 30, 40, 50, 100, 200, 300, 400, 500, 1000, 2000, 3000, 4000, 5000, 10000, or more regions) of the reference sequence other than the genetic locus comprising CYP21A2 gene and CYP21A1P pseudogene.

The method 500 proceeds from block 512 to block 516, where the computing system determines a total copy number of CYP21A2 gene and CYP21A1P pseudogene using a Gaussian mixture model comprising a plurality of Gaussians each representing a different integer copy number, given the number (e.g., normalized and/or corrected number) of the sequence reads aligned to CYP21A2 gene or CYP21A1P pseudogene. The computing system can determine the total copy number of CYP21A2 gene and CYP21A1P pseudogene using the Gaussian mixture model, given the normalized number of the sequence reads aligned to CYP21A2 gene or CYP21A1P pseudogene. The computing system can determine the total copy number of CYP21A2 gene and CYP21A1P pseudogene using the Gaussian mixture model, given the normalized, corrected number of the sequence reads aligned to CYP21A2 gene or CYP21A1P pseudogene.

The total copy number can be, for example, 2, 3, 4, 5, 6, 7, 8, 9, 10, or more. The Gaussian mixture model can comprise a one-dimensional Gaussian mixture model. The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers 0 to 10. A mean of each of the plurality of Gaussians can be the integer copy number represented by the Gaussian. A mean of each of the plurality of Gaussians can be the integer copy number represented by the Gaussian (e.g., copy numbers of 1, 2, 3, 4, 5, 6, 7, 8, 9, 10, 11, 12, 13, 14, 15, or more). The standard deviation of a Gaussian can be or be about, for example, 0.1, 0.2, 0.3, 0.4, 0.5, 0.6, 0.7, 0.8, 0.9, 1, or more. The plurality of Gaussians of the Gaussian mixture model can comprise, for example, 5, 6, 7, 8, 9, 10, 11, 12, 13, 14, 15, 16, or more, Gaussians. For example, the plurality of Gaussians of the Gaussian mixture model can comprise 5 Gaussians.

The computing system can determine the total copy number of CYP21A2 gene and CYP21A1P pseudogene using a Gaussian mixture model and a predetermined posterior probability threshold, given the normalized number of the sequence reads aligned to CYP21A2 gene or CYP21A1P pseudogene. The predetermined posterior probability threshold can be or be about, for example, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or more 0.7, 0.75, 0.8, 0.85, 0.95, or more. For example, the predetermined posterior probability threshold is 0.95.

The method 500 proceeds from block 516 to block 520, where the computing system phases one or more haplotypes originating from CYP21A2 gene or CYP21A1P pseudogene in a region of CYP21A2 gene, or a corresponding region of CYP21A1P pseudogene, comprising a plurality of CYP21A2/CYP21A1P differentiating bases (or positions or sites of differentiating bases) using sequence reads of the second plurality of sequence reads aligned to the region, or the corresponding region, comprising the plurality of CYP21A2/CYP21A1P differentiating bases. For example, a sequence read can be aligned to the reference sequence such that the sequence read overlaps a CYP21A2/CYP21A1P differentiating base (or the site of the CYP21A2/CYP21A1P differentiating base) or a base of the sequence read is aligned to the CYP21A2/CYP21A1P paralog differentiating base (or the site of the CYP21A2/CYP21A1P paralog differentiating base). A sequence read of the second plurality of sequence reads is aligned to the region of CYP21A2 gene, or the corresponding region of CYP21A1P pseudogene, comprising the plurality of CYP21A2/CYP21A1P differentiating bases with an alignment quality score of zero or more. A haplotype can comprise a reciprocal recombinant variant. A haplotype can comprise a non-reciprocal recombinant variant or a gene conversion variant.

The one or more haplotypes can comprise a wildtype CYP21A2 haplotype, a wildtype CYP21A1P, and/or a CYP21A2/CYP21A1P hybrid haplotype. A CYP21A2/CYP21A1P hybrid haplotype can include both CYP21A2 bases and CYP21A1P bases. A CYP21A2/CYP21A1P hybrid haplotype can be a recombinant variant. The CYP21A2/CYP21A1P hybrid haplotype can comprise a CYP21A2 variant haplotype or a CYP21A1P variant haplotype.

To phase the one or more haplotypes originating from CYP21A2 gene or CYP21A1P pseudogene, the computing system can analyze linkage information between CYP21A2/CYP21A1P differentiating bases of the plurality of CYP21A2/CYP21A1P differentiating bases using sequence reads of the second plurality of sequence reads aligned to the region, or the corresponding region, comprising the plurality of CYP21A2/CYP21A1P differentiating bases. The computing system can phase the one or more haplotypes originating from CYP21A2 gene or CYP21A1P pseudogene using sequence reads of the second plurality of sequence reads each aligned two or more of the plurality of CYP21A2/CYP21A1P differentiating bases. For example, referring to FIG. 1, assuming gene A and gene B shown in the figure are CYP21A2 gene or CYP21A1P pseudogene, respectively, one read pair that covers Site 1 and Site 4 can indicate the haplotype (haplotype x) the read pair originates from has CYP21A2 gene base at Site 1 and CYP21A2 gene base at Site 4. One read pair that covers Site 3 and Site 5 can indicate the haplotype (haplotype y) the read pair originates from has CYP21A2 gene base at Site 3 and CYP21A1P pseudogene base at Site 5. One read that covers Site 4 and Site 5 can indicate the haplotype (haplotype y) the read originates from has CYP21A2 gene base at Site 4 and CYP21A1P pseudogene base at Site 5. The computing system can phase all the haplotypes originating from either CYP21A2 gene or CYP21A1P pseudogene in the region with the set of five reliable base differences and identify haplotypes of CYP21A2 gene (haplotype 1) and CYP21A1P pseudogene (haplotype 2) and hybrid haplotypes (haplotypes 3 and 4). The number of haplotypes, the bases of the haplotypes at the sites, the number of sites, and the sites (e.g., Sites 1 and 4, or Sites 3 and 5) sequence reads cover are shown in FIG. 1 for illustration only and are not intended to be limiting.

The plurality of CYP21A2/CYP21A1P differentiating bases can comprise 14 (or 11, 12, 13, 15, 16, 17, or more) CYP21A2/CYP21A1P differentiating bases. The 14 CYP21A2/CYP21A1P differentiating bases can comprise 9 (or 6, 7, 8, 10, 11, 12, or more) CYP21A2/CYP21A1P recombinant variants. The CYP21A2/CYP21A1P differentiating bases can comprise chr6:32039081/32006353, 32039128/32006400, 32039132/32006404, 32039143/32006407, 32039426/32006690, 32039548/32006812, 32039802/32007066, 32039807/32007071, 32039810/32007074, 32039816/32007080, 32040182/32007446, 32040216/32007481, 32040421/32007686, and 32040535/32007800 of hg38, or corresponding bases thereof of a reference human genome sequence.

The method 500 proceeds from block 520 to block 524, where the computing system determines a copy number of each of the one or more haplotypes using the total copy number of CYP21A2 gene and CYP21A1P pseudogene and a number of sequence reads of the second plurality of sequence reads each comprising one or more of the plurality of CYP21A2/CYP21A1P differentiating bases that support the haplotype. The copy number of a haplotype can be, for example, 1, 2, 3, 4 or more.

The computing system can determine a CYP21A2 status (e.g., carrier, compound heterozygous, or homozygous) of the subject using the one or more haplotypes originating from CYP21A2 gene or CYP21A1P pseudogene in the region of CYP21A2 gene, or the corresponding region of CYP21A1P pseudogene, and/or the copy number of each of the one or more haplotypes. The computing system can generate a user interface (UI), such as a graphical user interface, comprising a UI element representing or comprising the CYP21A2 status. The UI can comprise the CYP21A2 status as a part of a UI element. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

Carrier. To determine the copy number of each of the one or more haplotypes, the computing system can determine a likelihood of one copy of a wildtype CYP21A2 haplotype is higher than a likelihood of two copies of the wildtype CYP21A2 haplotype given the number of sequence reads of the second plurality of sequence reads each comprising one or more of the plurality of CYP21A2/CYP21A1P differentiating bases that support the wildtype CYP21A2 haplotype. The computing system can determine the likelihood of one copy of the wildtype CYP21A2 haplotype is higher than the likelihood of two copies of the wildtype CYP21A2 haplotype for (e.g., using the sequence reads of) one, one or more (e.g., two, three, or four), or each of the one or more haplotypes. The likelihood difference can be, for example, 1%, 2%, 3%, 5%, 10%, 15%, 20%, or more. The computing system can determine the copy number of the wildtype CYP21A2 haplotype is one. to the computing system can determine the likelihood of one copy of the wildtype CYP21A2 haplotype is higher than the likelihood of two copies of the wildtype CYP21A2 haplotype for each of one or more pairs (or all pairs) of consecutive CYP21A2/CYP21A1P differentiating bases of the plurality of CYP21A2/CYP21A1P differentiating bases for which a first haplotype of the one or more haplotypes comprises CYP21A2 bases at the consecutive CYP21A2/CYP21A1P differentiating bases and a second haplotype of the one or more haplotypes comprises a CYP21A2 base and a CYP21A1P base (or a CYP21A1P base and a CYP21A2 base) at the consecutive CYP21A2/CYP21A1P differentiating bases. The first haplotype can comprise the CYP21A2 bases at the consecutive CYP21A2/CYP21A1P differentiating bases. The second haplotype can comprise a transition from the CYP21A2 base to the CYP21A1P base (or from the CYP21A1P base to the CYP21A2 base) between the consecutive the CYP21A2/CYP21A1P differentiating bases. Consecutive CYP21A2/CYP21A1P differentiating bases are consecutive within the plurality of CYP21A2/CYP21A1P differentiating bases, whether the consecutive CYP21A2/CYP21A1P differentiating bases are adjacent bases in the reference sequence. The computing system can combine (e.g., averaging or weighted averaging) the likelihoods determined for each of one or more pairs (or all pairs) of consecutive CYP21A2/CYP21A1P differentiating bases to determine the likelihood of one copy of the wildtype CYP21A2 haplotype is higher than the likelihood of two copies of the wildtype CYP21A2 haplotype.

The computing system can determine the likelihood of one copy of the wildtype CYP21A2 haplotype is higher than the likelihood of two copies of the wildtype CYP21A2 haplotype for each of one or more pairs (or all pairs) of consecutive CYP21A2/CYP21A1P differentiating bases given (1) a number of sequence reads of the second plurality of sequence reads each comprising the CYP21A2 bases at the consecutive CYP21A2/CYP21A1P differentiating bases. The computing system can determine the likelihood of one copy of the wildtype CYP21A2 haplotype is higher than the likelihood of two copies of the wildtype CYP21A2 haplotype for each of one or more pairs (or all pairs) of consecutive CYP21A2/CYP21A1P differentiating bases given (2) a number of sequence reads of the second plurality of sequence reads each comprising the CYP21A2 base and the CYP21A1P base at the consecutive CYP21A2/CYP21A1P differentiating bases, and/or (3) a number of sequence reads of the second plurality of sequence reads each comprising the CYP21A1P base and the CYP21A2 base at the consecutive CYP21A2/CYP21A1P differentiating bases. The computing system can determine the likelihood of one copy of the wildtype CYP21A2 haplotype is higher than the likelihood of two copies of the wildtype CYP21A2 haplotype for each of one or more pairs (or all pairs) of consecutive CYP21A2/CYP21A1P differentiating bases given (4) a number of sequence reads of the second plurality of sequence reads each comprising the CYP21A1P bases at the consecutive CYP21A21CYP21A1P differentiating bases.

The copy number of the wildtype CYP21A2 haplotype can be one. The computing system can determine the subject is a carrier of a CYP21A2 variant haplotype. The one or more haplotypes can comprise four haplotypes. The total copy number of CYP21A2 gene and CYP21A1P gene can be four. The copy number of each of the four haplotypes can be one (e.g., one copy of wildtype CYP21A2 haplotype, one copy of a CYP21A2 variant haplotype, one copy of CYP21A1P wildtype haplotype, and one copy of a haplotype with CYP21A1P bases at a high percentage (such as 80%, 85%, 90%, 95%, or more) of the differentiating bases and thus is unlikely a CYP21A2 variant haplotype/is likely a CYP21A1P variant haplotype. The computing system can determine the CYP21A2 status of the subject as a carrier of a CYP21A2 variant haplotype. The one or more haplotypes can comprise three haplotypes. The total copy number of CYP21A2 gene and CYP21A1P gene can be four. The copy number of the wildtype CYP21A2 haplotype, the CYP21A2 variant haplotype, and the CYP21A1P wildtype haplotype (or a haplotype with CYP21A1P bases at a high percentage of the differentiating bases, such as 80%, 85%, 90%, 95%, or more) can be one, one, and two, respectively. The computing system can determine the subject is a carrier of a CYP21A2 variant haplotype.

Compound heterozygous. The one or more haplotypes can comprise two or more haplotypes. None of the two or more haplotypes may comprise CYP21A2 base at each of the plurality of CYP21A2/CYP21A1P differentiating bases. None of the two or more haplotypes may comprise CYP21A2 bases at all of the plurality of CYP21A2/CYP21A1P differentiating bases. Each of the two or more haplotypes may comprise a CYP21A1P base at one or more of the plurality of CYP21A2/CYP21A1P differentiating bases. The computing system can determine the subject is a compound heterozygous of CYP21A2 variant haplotypes.

Homozygous. The one or more haplotypes can comprise an identical base (e.g., a CYP21A base or a CYP21A1P base) at a CYP21A2/CYP21A1P differentiating base or at each of two or more of the plurality of CYP21A2/CYP21A1P differentiating bases. The computing system can determine the subject is homozygous (e.g., homozygous for the wildtype CYP21A1P gene haplotype or homozygous of a CYP21A2 gene variant) at one or more of the plurality of the CYP21A2/CYP21A1P differentiating bases.

The one or more haplotypes can comprise only one haplotype. The only one haplotype can comprise no CYP21A2 base at one, one or more, or each of the plurality of CYP21A2/CYP21A1P differentiating bases. The computing system can determine the subject is homozygous of a CYP21A2 variant haplotype. For example, based on the plurality of CYP21A2/CYP21A1P differentiating bases, the computing system can determine the copy number (CN) of a CYP21A2 base. Using the number of reads supporting a CYP21A2 base or a CYP21A1P gene base, as well as the total CN of the CYP21A2 gene and the CYP21A1P gene, the most likely combination of the CN of the CYP21A2 base and the CN of the CYP21A1P gene base can be determined. If the CN of the CYP21A2 base is determined as 0, this indicates that the subject has no copy of the wildtype CYP21A2 gene haplotype (the haplotype that carries the CYP21A2 gene base at a differentiating base), and is homozygous for the CYP21A2 gene variant haplotype.

The method 500 ends at block 528.

### Determining Gene Recombinant Variants and Gene Variant Status

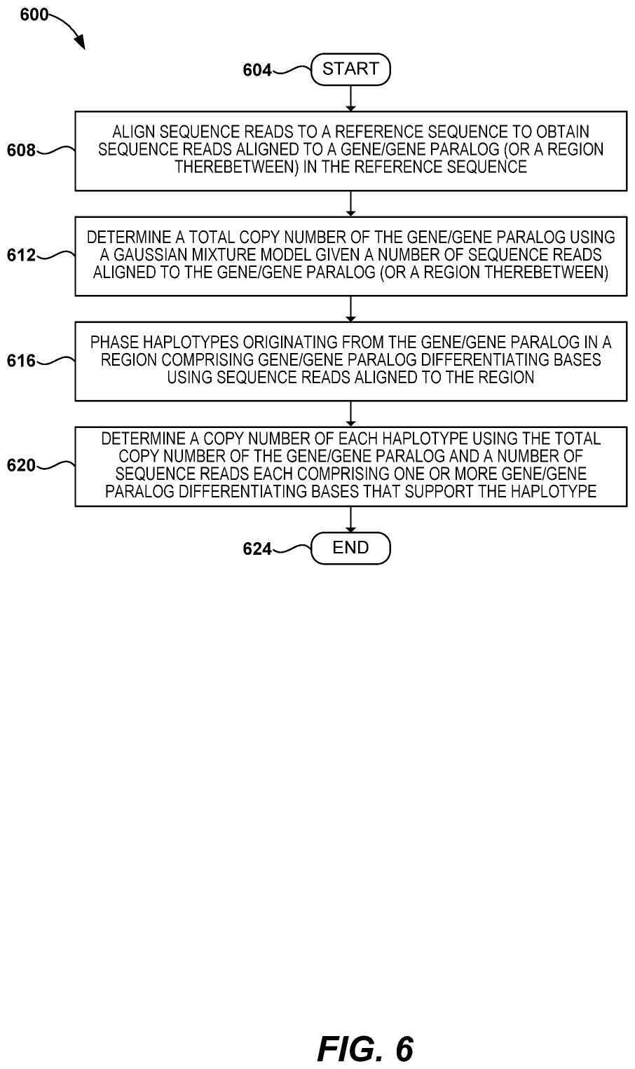

FIG. 6 is a flow diagram showing an exemplary method 600 of determining or identifying one or more gene recombinant variants or gene variant status. The method 600 may be embodied in a set of executable program instructions stored on a computer-readable medium, such as one or more disk drives, of a computing system. For example, the computing system 700 shown in FIG. 7 and described in greater detail below can execute a set of executable program instructions to implement the method 600. When the method 600 is initiated, the executable program instructions can be loaded into memory, such as RAM, and executed by one or more processors of the computing system 700. Although the method 600 is described with respect to the computing system 700 shown in FIG. 7, the description is illustrative only and is not intended to be limiting. In some embodiments, the method 600 or portions thereof may be performed serially or in parallel by multiple computing systems.

After the method 600 begins at block 604, the method 600 proceeds to block 608, where a computing system (such as the computing system 700 described with reference to FIG. 7) aligns a first plurality of sequence reads to a reference sequence (e.g., a reference genome sequence, such as hg19, or hg38) to obtain a second plurality of sequence reads aligned to a gene or a gene paralog (or a region therebetween) in the reference sequence (including alignment of each of the second plurality of sequence reads to the gene or the gene paralog in the reference sequence). The computing system can receive the first plurality of sequence reads generated from a sample obtained from a subject. The computing system can store the first plurality of sequence reads in memory. The computing system can load the first plurality of sequence reads into memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, Calif.).

Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by whole genome sequencing (WGS). The WGS can be clinical WGS (cWGS). The sample can comprise cells, cell-free DNA, cell-free fetal DNA, amniotic fluid, a blood sample, a biopsy sample, or a combination thereof.

A sequence read can be aligned to the gene or the pseudogene in the reference sequence with an alignment quality score of zero or more. A sequence read can be aligned to the gene or the pseudogene in the reference sequence with an alignment quality score of about zero (e.g., when a sequence is aligned to a region where the gene and the gene paralog are highly homologous). The computing system can align sequence reads to the reference sequence using an aligner or an alignment method such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The gene paralog can be a gene. The gene paralog can be a pseudogene. The gene and the gene paralog have a sequence identity of at least 70%, 75%, 80%, 85%, 90%, 95%, 96%, 97%, 98%, 99%, or more. In some embodiments, the gene is GBA gene, and the gene paralog is GBAP1 gene. If the gene is GBA gene, and the gene paralog is GBAP1 gene, the computing system can perform the method 400 (or one or more steps of method 400) described with reference to FIG. 4. In some embodiments, the gene is CYP21A2 gene, and the gene paralog is CYP21A1P pseudogene. If the gene is CYP21A2 gene, and the gene paralog is CYP21A1P pseudogene, the computing system can perform the method 500 (or one or more steps of method 500) described with reference to FIG. 5. In some embodiments, the gene is ABCC6, ABCD1, ACTB, ACTG1, ACTN4, ADAMTSL2, ADIPOR1, AFG3L2, AGK, ALG1, ALMS1, ANKRD11, ANOS1, AP4S1, ARM4, ARSE, ASNS, ATAD3A, B3GAT3, BCAP31, BDP1, BMPR1A, BRAF, BRCA1, C2, CACNAIC, CALM1, CD46, CEP290, CFH, CFH, CFH, CHEK2, CISD2, CLCNKA, CLCNKB, CORO1A, COX10, CP, CRYBB2, CSF2RA, CUBN, CUBN, CYCS, CYPIIB1, CYP21A2, DCLRE1C, DHFR, DICER1, DIS3L2, DNAH11, DNAH11, DNM1, DSE, DUOX2, EGLN1, ELK1, ELMO2, ERCC6, ESPN, EYS, F8, FANCD2, FANCD2, FAR1, FHL1, FLG, FLNC, FOXD4, FXN, GBA, GH1, GK, GLUD1, GLUD1, GOSR2, GUSB, HBA1, HBA2, HNRNPA1, HPS1, HSPD1, HYDIN, IDS, IFT122, IGLL1, KANSL1, KCTD1, KIF1C, KRAS, KRT14, KRT16, KRT17, KRT6A, KRT6B, KRT6C, LEFTY2, LRP5, LRP5, MAT2A, MID1, MOCS1, MSN, MSX2, MYO5B, NCF1, NEB, NECAP1, NEFH, NF1, NF1, NF1, NOTCH2, NXF5, OCLN, OTOA, PARN, PBX1, PIGA, PIGN, PIK3CA, PIK3CD, PKD1, PKP2, PMS2, PMS2, PMS2, PNPT1, POLH, PRODH, PRODH, PROS1, PRPS1, PRSS1, PTEN, RAD21, RBM8A, RBPJ, RDX, RMND1, RNF216, RNF216, RPL15, SALL1, SBDS, SDHA, SHOX, SLC25A15, SLC25A15, SLC33A1, SLC6A8, SMN1, SMN2, SOX2, SPTLC1, SRD5A3, SRP72, STAT5B, STRC, SYT14, TARDBP, TBL1XR1, TBX20, TIMM8A, TPM3, TPMT, TRAPPC2, TRIP11, TTN, TUBA1A, TUBB2A, TUBB2B, TUBB3, TUBB4A, TUBG1, TYR, UBA5, UBE3A, UNC93B1, USP18, VPS35, VWF, WRN, XIAP, ZEB2, or ZNF341.

In some embodiments, the computing system can determine a number of the sequence reads aligned to the gene or the gene paralog (or a region therebetween). The number of the sequence reads aligned to the gene or the gene paralog (or a region therebetween) comprises a normalized and/or GC corrected number of the sequence reads aligned to the gene or the gene paralog (or a region therebetween).

The computing system can determine the normalized number of the sequence reads aligned to the gene or the gene paralog in the reference sequence using (1a) a depth of the sequence reads aligned to the gene or the gene paralog, (1b) a length of the unique region, (2a) a depth of sequence reads of the first plurality of sequence reads aligned to each of a plurality of regions of the reference sequence other than a genetic locus comprising the gene and the gene paralog, and/or (2b) a length of each of the plurality of regions of the reference other than the genetic locus comprising the gene and the gene paralog. The computing system can determine a normalized, corrected number of the sequence reads aligned to the gene or the gene paralog in the reference sequence from the normalized number of the sequence reads aligned to the gene or the gene paralog in the reference sequence. To determine the normalized, corrected number of the sequence reads aligned to the gene or the gene paralog in the reference sequence, the computing system can determine a normalized, GC content-corrected number of the sequence reads aligned to the gene or the gene paralog in the reference sequence from the normalized number of the sequence reads aligned to the gene or the gene paralog in the reference sequence. The computing system can determine the normalized, GC content-corrected number of the sequence reads aligned to the gene or the gene paralog in the reference sequence from the normalized number of the sequence reads aligned to the gene or the gene paralog in the reference sequence using (1) a GC content of the gene or the gene paralog. The computing system can determine the normalized, GC content-corrected number of the sequence reads aligned to the gene or the gene paralog in the reference sequence from the normalized number of the sequence reads aligned to the gene or the gene paralog in the reference sequence using (1) a GC content of the gene or the gene paralog and/or (2) a GC content of each of one or more regions of the reference sequence other than a genetic locus comprising the gene and the gene paralog (or one or more regions of the reference sequence not comprising the gene and the gene paralog). For example, the computing system can determine the normalized, GC content-corrected number of the sequence reads aligned to the gene or the gene paralog in the reference sequence from the normalized number of the sequence reads aligned to the gene or the gene paralog in the reference sequence using (1) the GC content of the gene or the gene paralog and (2) the GC content of one region of the reference sequence other than the genetic locus comprising the gene and the gene paralog. As another example, the computing system can determine the normalized, GC content-corrected number of the sequence reads aligned to the gene or the gene paralog in the reference sequence from the normalized number of the sequence reads aligned to the gene or the gene paralog in the reference sequence using (1) the GC content of the gene or the gene paralog and (2) the GC contents of multiples regions (e.g., 2, 3, 4, 5, 10, 20, 30, 40, 50, 100, 200, 300, 400, 500, 1000, 2000, 3000, 4000, 5000, 10000, or more regions) of the reference sequence other than the genetic locus comprising the gene and the gene paralog.

The method 600 proceeds from block 608 to block 612, where the computing system determines a total copy number of the gene and the gene paralog using a Gaussian mixture model comprising a plurality of Gaussians each representing a different integer copy number, given a number of the sequence reads aligned to the gene or the gene paralog (or a region therebetween). The computing system can determine the total copy number of the gene and the gene paralog using the Gaussian mixture model, given the number of the sequence reads (e.g., normalized and/or corrected number of the sequence reads) aligned to the gene or the gene paralog.

The total copy number can be, for example, 2, 3, 4, 5, 6, 7, 8, 9, 10, or more. The Gaussian mixture model can comprise a one-dimensional Gaussian mixture model. The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers 0 to 10. A mean of each of the plurality of Gaussians can be the integer copy number represented by the Gaussian. A mean of each of the plurality of Gaussians can be the integer copy number represented by the Gaussian (e.g., copy numbers of 1, 2, 3, 4, 5, 6, 7, 8, 9, 10, 11, 12, 13, 14, 15, or more). The standard deviation of a Gaussian can be or be about, for example, 0.1, 0.2, 0.3, 0.4, 0.5, 0.6, 0.7, 0.8, 0.9, 1, or more. The plurality of Gaussians of the Gaussian mixture model can comprise, for example, 5, 6, 7, 8, 9, 10, 11, 12, 13, 14, 15, 16, or more, Gaussians. For example, the plurality of Gaussians of the Gaussian mixture model can comprise 5 Gaussians.

To determine the total copy number of the gene and the gene paralog, the computing system can determine a copy number of a region between the gene or the gene paralog using the Gaussian mixture model, given the normalized number of the sequence reads aligned to the gene or the gene paralog. The total copy number of the gene and the gene paralog can be the copy number of the region between the gene or the gene paralog plus two.

The computing system can determine the total copy number of the gene and the gene paralog using a Gaussian mixture model and a predetermined posterior probability threshold, given the normalized number of the sequence reads aligned to the gene or the gene paralog. The predetermined posterior probability threshold can be, or be about, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or more. For example, the predetermined posterior probability threshold is 0.95.

The method 600 proceeds from block 612 to block 616, where the computing system phases one or more haplotypes of or originating from the gene, comprising a recombinant variant of the gene, or the gene paralog, or a region of the gene or a corresponding region of the gene paralog, comprising a plurality of gene/gene paralog differentiating bases (or positions or sites of differentiating bases) using sequence reads of the second plurality of sequence reads aligned to the region, or the corresponding region, comprising the plurality of gene/gene paralog differentiating bases. For example, a sequence read can be aligned to the reference sequence such that the sequence read overlaps a gene/gene paralog differentiating base (or the site of the gene/gene paralog differentiating base) or a base of the sequence read is aligned to the gene/gene paralog differentiating base (or the site of the gene/gene paralog differentiating base). A sequence read can be aligned to the reference sequence of the second plurality of sequence reads can be aligned to the region of the gene, or the corresponding region of the gene paralog, comprising the plurality of gene/gene paralog differentiating bases with an alignment quality score of zero or more.

The one or more haplotypes can comprise a wildtype gene haplotype, a wildtype gene paralog, and/or a gene/gene paralog hybrid haplotype. A gene/gene paralog hybrid haplotype can include both gene bases and gene paralog bases. A gene/gene paralog hybrid haplotype can be a recombinant variant. The gene/gene paralog hybrid haplotype can comprise a gene variant haplotype or a gene paralog variant haplotype. The gene recombinant variant can comprise a reciprocal recombinant variant. The gene recombinant variant can comprise a non-reciprocal recombinant variant or a gene conversion variant.

To phase the one or more haplotypes originating from the gene or the gene paralog, the computing system can analyze linkage information between gene/gene paralog differentiating bases of the plurality of the gene/gene paralog differentiating bases using sequence reads of the second plurality of sequence reads aligned to the region, or the corresponding region, comprising the plurality of gene/gene paralog differentiating bases. The computing system can phase the one or more haplotypes originating from the gene or the gene paralog using sequence reads of the second plurality of sequence reads each aligned two or more of the plurality of gene/gene paralog differentiating bases. For example, referring to FIG. 1, assuming gene A and gene B shown in the figure are the gene or the gene paralog, respectively, one read pair that covers Site 1 and Site 4 can indicate the haplotype (haplotype x) the read pair originates from has the gene base at Site 1 and the gene base at Site 4. One read pair that covers Site 3 and Site 5 can indicate the haplotype (haplotype y) the read pair originates from has the gene base at Site 3 and the gene paralog base at Site 5. One read that covers Site 4 and Site 5 can indicate the haplotype (haplotype y) the read originates from has the gene base at Site 4 and the gene paralog base at Site 5. The caller can phase all the haplotypes originating from either the gene or the gene paralog in the region with the set of five reliable base differences and identify haplotypes of the gene (haplotype 1) and the gene paralog (haplotype 2) and hybrid haplotypes (haplotypes 3 and 4). The number of haplotypes, the bases of the haplotypes at the sites, the number of sites, and the sites (e.g., Sites 1 and 4, or Sites 3 and 5) sequence reads cover are shown in FIG. 1 for illustration only and are not intended to be limiting.

The method 600 proceeds from block 616 to block 620, where the computing system determines a copy number of each of the one or more haplotypes using the total copy number of the gene and the gene paralog and a number of sequence reads of the second plurality of sequence reads each comprising one or more of the plurality of gene/gene paralog differentiating bases that support the haplotype. The copy number of a haplotype can be, for example, 1, 2, 3, 4 or more. The computing system can determine a gene variant status (e.g., carrier, compound heterozygous, or homozygous) of the subject using the one or more haplotypes originating from the gene or the gene paralog, or the region of the gene or the corresponding region of the gene paralog, and/or the copy number of each of the one or more haplotypes. The computing system can generate a user interface (UI), such as a graphical user interface, comprising a UI element representing or comprising the gene variant status. The UI can comprise the status of the gene variant as a part of a UI element. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

Carrier. To determine the copy number of each of the one or more haplotypes, the computing system can determine a likelihood of one copy of a wildtype gene haplotype is higher than a likelihood of two copies of the wildtype gene haplotype given the number of sequence reads of the second plurality of sequence reads each comprising one or more of the plurality of gene/gene paralog differentiating bases that support the wildtype gene haplotype. The computing system can determine the likelihood of one copy of the wildtype gene haplotype is higher than the likelihood of two copies of the wildtype gene haplotype for (e.g., using the sequence reads of) one, one or more (e.g., two, three, or four), or each of the one or more haplotypes. The likelihood difference can be, for example, 1%, 2%, 3%, 5%, 10%, 15%, 20%, or more. The computing system can determine the copy number of the wildtype gene haplotype is one. If the likelihood of one copy of the wildtype gene haplotype is higher than the likelihood of two copies of the wildtype gene haplotype, the copy number of the wildtype gene haplotype can be one.

In some embodiments, the computing system can determine the likelihood of one copy of the wildtype gene haplotype is higher than the likelihood of two copies of the wildtype gene haplotype for each of one or more pairs (or all pairs) of consecutive gene/gene paralog differentiating bases of plurality of gene/gene paralog differentiating bases for which a first haplotype of the one or more haplotypes comprises gene bases at the consecutive gene/gene paralog differentiating bases and a second haplotype of the one or more haplotypes comprises a gene base and a gene paralog base (or a gene paralog base and a gene base) at the consecutive gene/gene paralog differentiating bases.

The first haplotype can comprise the gene bases at the consecutive gene/gene paralog differentiating bases. The second haplotype can comprise a transition from the gene base to the gene paralog base (or from the gene paralog base to the gene base) between the consecutive gene/gene paralog differentiating bases. Consecutive gene/gene paralog differentiating bases are consecutive within the plurality of gene/gene paralog differentiating bases, whether the consecutive gene/gene paralog differentiating bases are adjacent bases in the reference sequence. For example, gene/gene paralog differentiating bases at Site 2 and Site 3 (or positions of differentiating bases) in the examples below are consecutive gene/gene paralog differentiating bases, whether the consecutive gene/gene paralog differentiating bases are adjacent bases in the reference sequence. The computing system can combine (e.g., averaging or weighted averaging) the likelihoods determined for each of one or more pairs (or all pairs) of consecutive gene/gene paralog differentiating bases to determine the likelihood of one copy of the wildtype gene haplotype is higher than the likelihood of two copies of the wildtype gene haplotype. The likelihood of one copy of the wildtype gene haplotype can comprise an aggregate of the likelihood of one copy of the wildtype gene haplotype determined for each of the one or more pairs of consecutive gene/gene paralog differentiating bases. The likelihood of two copies of the wildtype gene haplotype can comprise an aggregate of the likelihood of two copies of the wildtype gene haplotype determined for each of the one or more pairs of consecutive gene/gene paralog differentiating bases.

The computing system can determine the likelihood of one copy of the wildtype gene haplotype is higher than the likelihood of two copies of the wildtype gene haplotype for one or more (or all) pairs of consecutive gene/gene paralog differentiating bases given (1) a number of sequence reads of the second plurality of sequence reads each comprising the gene bases at the consecutive gene/gene paralog differentiating bases. The computing system can determine the likelihood of one copy of the wildtype gene haplotype is higher than the likelihood of two copies of the wildtype gene haplotype for one or more (or all) pairs of consecutive gene/gene paralog differentiating bases given (2) a number of sequence reads of the second plurality of sequence reads each comprising the gene base and the gene paralog base at the consecutive gene/gene paralog differentiating bases, and/or a number of sequence reads of the second plurality of sequence reads each comprising the gene paralog base and the gene base at the consecutive gene/gene paralog differentiating bases. In some embodiments, computing system can determine the likelihood of one copy of the wildtype gene haplotype is higher than the likelihood of two copies of the wildtype gene haplotype for each of one or more pairs (or all pairs) of consecutive gene/gene paralog differentiating bases given (4) a number of sequence reads of the second plurality of sequence reads each comprising the gene paralog bases at the consecutive gene/gene paralog differentiating bases.

For example, by analyzing linkage information between gene/gene paralog differentiating bases, the following haplotypes (at six sites for illustrative purposes only) can be determined for a subject:

| | Site/Differentiating Base | | | | | |
|---|---|---|---|---|---|---|
| Haplotype | 1 | 2 | 3 | 4 | 5 | 6 |
| 1 | gene | gene | gene | gene | gene | gene |
| 2 | gene | gene | paralog | gene | gene | gene |
| 3 | paralog | paralog | paralog | paralog | paralog | paralog |

A transition from the gene base to the gene paralog base occurs between Site 2 and Site 3 for haplotype 2. For example, the number of reads with the gene bases at Site 2 and Site 3 is 103, the number of reads with the gene base at Site 2 and the gene paralog base at Site 3 is 99, and the number of reads with the gene paralog bases at Site 2 and Site 3 is 210. The computing system can determine the likelihood of one copy of the wildtype gene haplotype is higher than the likelihood of two copies of the wildtype gene haplotype given the number of reads with the gene bases at Site 2 and Site 3 is 103 and the number of reads with the gene base at Site 2 and the gene paralog base at Site 3 is 99. The computing system can determine the likelihood of one copy of the wildtype gene haplotype is higher than the likelihood of two copies of the wildtype gene haplotype without using the reads from the wildtype gene paralog haplotype.

Continuing with the example, a transition from the gene base to the gene paralog base occurs between Site 3 and Site 4 for haplotype 2. For example, the number of reads with the gene bases at Site 3 and Site 4 is 100, the number of reads with the gene paralog base at Site 3 and the paralog base at Site 4 is 99, and the number of reads with the gene paralog bases at Site 3 and Site 4 is 190. The computing system can determine the likelihood of one copy of the wildtype gene haplotype is higher than the likelihood of two copies of the wildtype gene haplotype given the number of reads with the gene bases at Site 3 and Site 4 is 100 and the number of reads with the gene paralog base at Site 3 and the gene base at Site 4 is 99. The computing system can determine the likelihood of one copy of the wildtype gene haplotype is higher than the likelihood of two copies of the wildtype gene haplotype without using the reads from the wildtype gene paralog haplotype.

In some embodiments, the computing system can determine the likelihood of one copy of the wildtype gene haplotype is higher than the likelihood of two copies of the wildtype gene haplotype by combining (e.g., averaging or weighted averaging) (1) the likelihood of one copy of the wildtype gene haplotype is higher than the likelihood of two copies of the wildtype gene haplotype determined given the number of reads with the gene bases at Site 2 and Site 3 and the number of reads with the gene base at Site 2 and the gene paralog base at Site 3 and (2) the likelihood of one copy of the wildtype gene haplotype is higher than the likelihood of two copies of the wildtype gene haplotype determined given the number of reads with the gene bases at Site 3 and 4 and the number of reads with the gene paralog base at Site 3 and the gene base at Site 4.

In some embodiments, the computing system can determine the likelihood of one copy of the wildtype gene haplotype is higher than the likelihood of two copies of the wildtype gene haplotype given (1) the total number of sequence reads for each of one or more pairs (or all pairs) of consecutive gene/gene paralog differentiating bases of plurality of gene/gene paralog differentiating bases for which a first haplotype of the one or more haplotypes comprises gene bases at the consecutive gene/gene paralog differentiating bases. The computing system can determine the likelihood of one copy of the wildtype gene haplotype is higher than the likelihood of two copies of the wildtype gene haplotype based on and (2) the total number of sequence reads for each of one or more pairs (or all pairs) of consecutive gene/gene paralog differentiating bases of plurality of gene/gene paralog differentiating bases for which a second haplotype of the one or more haplotypes comprises a gene base and a gene paralog base (or a gene paralog base and a gene base) at the consecutive gene/gene paralog differentiating bases. In the above example, the computing system can determine the likelihood of one copy of the wildtype gene haplotype is higher than the likelihood of two copies of the wildtype gene haplotype given (1) the total number of reads with the gene bases at Site 2 and Site 3 and reads with the gene base at Site 3 and Site 4 and (2) the number of reads with the gene base at Site 2 and the gene paralog base at Site 3 and reads with the gene paralog base at Site 3 and the gene base at Site 4.

For example, by analyzing linkage information between gene/gene paralog differentiating bases, the following haplotypes (at six bases or sites or positions of differentiating bases for illustrative purposes only) can be determined for a subject:

| | Site/Differentiating Base | | | | | |
|---|---|---|---|---|---|---|
| Haplotype | 1 | 2 | 3 | 4 | 5 | 6 |
| 1 | gene | gene | gene | gene | gene | gene |
| 2 | gene | gene | paralog | gene | gene | gene |
| 3 | paralog | paralog | gene | paralog | paralog | paralog |
| 4 | paralog | paralog | paralog | paralog | paralog | paralog |

A transition from the gene base to the gene paralog base occurs between Site 2 and Site 3 for haplotype 2. For example, the number of reads with the gene bases at Site 2 and 3 is 103, the number of reads with the gene base at Site 2 and the gene paralog base at Site 3 is 99, the number of reads with the gene paralog base at Site 2 and the gene base at Site 3 is 90, and the number of reads with the gene paralog bases at Site 2 and Site 3 is 104. The computing system can determine the likelihood of one copy of the wildtype gene haplotype is higher than the likelihood of two copies of the wildtype gene haplotype given the number of reads with the gene bases at Site 2 and Site 3 is 103, the number of reads with the gene base at Site 2 and the gene paralog base at Site 3 is 99. The computing system can determine the likelihood of one copy of the wildtype gene haplotype is higher than the likelihood of two copies of the wildtype gene haplotype without using the reads from the wildtype gene paralog haplotype. The computing system can determine the likelihood of one copy of the wildtype gene haplotype is higher than the likelihood of two copies of the wildtype gene haplotype without using the reads with the gene paralog base at Site 2 and the gene base at Site 3 because that haplotype (haplotype 3) has mostly gene paralog bases at the sites/positions of differentiating bases and thus is unlikely a gene variant haplotype/is likely to be a gene paralog variant haplotype.

The copy number of the wildtype gene haplotype can be one. The computing system can determine the subject is a carrier of a gene variant haplotype. The one or more haplotypes can comprise four haplotypes (e.g., one copy of wildtype gene haplotype, one copy of a gene variant haplotype, one copy of gene paralog wildtype haplotype, and one copy of a haplotype with gene paralog bases at a high percentage (such as 80%, 85%, 90%, 95%, or more) of the differentiating bases and thus is unlikely a gene variant haplotype/is likely a gene paralog variant haplotype. The total copy number of the gene and the gene paralog can be four. The copy number of each of the four haplotypes can be one. The computing system can determine the gene variant status of the subject as a carrier of a gene variant haplotype. For example, by analyzing linkage information between gene/gene paralog differentiating bases, the following haplotypes (at six sites for illustrative purposes only) can be determined for a subject:

| | No. of | Site/Differentiating Base | | | | | |
|---|---|---|---|---|---|---|---|
| Haplotype | Copy(ies) | 1 | 2 | 3 | 4 | 5 | 6 |
| 1 | 1 | gene | gene | gene | gene | gene | gene |
| 2 | 1 | gene | gene | paralog | paralog | gene | gene |
| 3 | 1 | paralog | paralog | paralog | gene | paralog | paralog |
| 4 | 1 | paralog | paralog | paralog | paralog | paralog | paralog |

The computing system can determine the likelihood of one copy of the wildtype gene haplotype bases at Site 2 and Site 3 is higher than the likelihood of two copies of the wildtype gene bases at Site 2 and Site 3 given the number of reads with the gene bases at Site 2 and Site 3 (haplotype 1) and the number of reads with the gene base at Site 2 and the gene paralog base at Site 3 (haplotype 2). The computing system can determine the likelihood of one copy of the wildtype gene haplotype bases at Site 4 and Site 5 is higher than the likelihood of two copies of the wildtype gene bases at Site 4 and Site 5 given the number of reads with the gene bases at Site 4 and Site 5 (haplotype 1) and the number of reads with the gene paralog base at Site 4 and the gene base at Site 5 (haplotype 2). The computing system can determine the likelihood of one copy of the wildtype gene haplotype is higher than the likelihood of two copies of the wildtype gene haplotype given the number of reads with the gene bases at Site 2 and Site 3 (haplotype 1) and the number of reads with the gene base at Site 2 and the gene paralog base at Site 3 (haplotype 2), and/or the number of reads with the gene bases at Site 4 and Site 5 (haplotype 1) and the number of reads with the gene paralog base at Site 4 and the gene base at Site 5 (haplotype 2). Because the subject has one copy of wildtype gene haplotype and one copy of a gene variant haplotype (and one copy of the wildtype gene paralog haplotype, and one copy of a haplotype with mostly gene paralog bases at the sites/positions of differentiating bases and thus is unlikely a gene variant haplotype/is likely a gene paralog variant haplotype), the subject is a carrier of a gene variant haplotype.

The one or more haplotypes can comprise three haplotypes. The total copy number of gene and gene paralog gene can be four. The copy number of the wildtype gene haplotype, the gene variant haplotype, and the wildtype gene paralog haplotype (or a haplotype with gene paralog bases at a high percentage of the differentiating bases, such as 80%, 85%, 90%, 95%, or more) can be one, one, and two, respectively. The computing system can determine the gene status of the subject as a carrier of a gene variant haplotype. For example, by analyzing linkage information between gene/gene paralog differentiating bases, the following haplotypes (at six sites for illustrative purposes only) can be determined for a subject:

| | No. of | Site/Differentiating Base | | | | | |
|---|---|---|---|---|---|---|---|
| Haplotype | Copy(ies) | 1 | 2 | 3 | 4 | 5 | 6 |
| 1 | 1 | gene | gene | gene | gene | gene | gene |
| 2 | 1 | gene | gene | paralog | paralog | gene | gene |
| 3 | 2 | paralog | paralog | paralog | paralog | paralog | paralog |

The computing system can determine the likelihood of one copy of the wildtype gene haplotype bases at Site 2 and Site 3 is higher than the likelihood of two copies of the wildtype gene bases at Site 2 and Site 3 given the number of reads with the gene bases at Site 2 and Site 3 (haplotype 1) and the number of reads with the gene base at Site 2 and the gene paralog base at Site 3 (haplotype 2). The computing system can determine the likelihood of one copy of the wildtype gene haplotype bases at Site 4 and Site 5 is higher than the likelihood of two copies of the wildtype gene bases at Site 4 and Site 5 given the number of reads with the gene bases at Site 4 and Site 5 (haplotype 1) and the number of reads with the gene paralog base at Site 4 and the gene base at Site 5 (haplotype 2). The computing system can determine the likelihood of one copy of the wildtype gene haplotype is higher than the likelihood of two copies of the wildtype gene haplotype given the number of reads with the gene bases at Site 2 and Site 3 (haplotype 1) and the number of reads with the gene base at Site 2 and the gene paralog base at Site 3 (haplotype 2), and/or the number of reads with the gene bases at Site 4 and Site 5 (haplotype 1) and the number of reads with the gene paralog base at Site 4 and the gene base at Site 5 (haplotype 2). Because the subject has one copy of wildtype gene haplotype and one copy of a gene variant haplotype, the subject is a carrier of a gene variant haplotype.

Compound heterozygous. The one or more haplotypes can comprise two or more haplotypes. None of the two or more haplotypes may comprise a gene base at each of the plurality of gene/gene paralog differentiating bases. None of the two or more haplotypes may comprise only gene bases at all of the plurality of gene/gene paralog differentiating bases. Each of the two or more haplotypes may comprise a gene paralog base at one or more of the plurality of gene/gene paralog differentiating bases. The computing system can determine the subject is a compound heterozygous of gene variant haplotypes. For example, by analyzing linkage information between gene/gene paralog differentiating bases, the following haplotypes (at six sites for illustrative purposes only) can be determined for a subject:

| | No. of | Site/Differentiating Base | | | | | |
|---|---|---|---|---|---|---|---|
| Haplotype | Copy(ies) | 1 | 2 | 3 | 4 | 5 | 6 |
| 1 | 1 | paralog | gene | gene | gene | gene | gene |
| 2 | 1 | gene | gene | paralog | paralog | gene | gene |
| 3 | 2 | paralog | paralog | paralog | paralog | paralog | paralog |

Because the subject does not have any copy of the wildtype gene haplotype and has one copy of each of two gene variant haplotypes, the subject is compound heterozygous of gene variant haplotypes.

Homozygous. The one or more haplotypes can comprise an identical base (e.g., a gene base or a gene paralog base) at a gene/gene paralog differentiating base or at each of two or more of the plurality of gene/gene paralog differentiating bases. The computing system can determine the subject is homozygous (e.g., homozygous for the wildtype gene paralog haplotype or homozygous for a gene variant haplotype) at one or more of the plurality of the gene/gene paralog differentiating bases.

The one or more haplotypes can comprise only one haplotype. The only one haplotype can comprise no gene base at one, one or more, or each of plurality of the gene/gene paralog differentiating bases. The only one haplotype can comprise gene paralog base at one, one or more, or each of plurality of the gene/gene paralog differentiating bases. The computing system can determine the subject is homozygous of a gene variant haplotype at one, one or more, or each of plurality of the gene/gene paralog differentiating bases. For example, based on the plurality of gene/gene paralog differentiating bases, the computing system can determine the copy number (CN) of a gene base. Using the number of reads supporting a gene base or a gene paralog base, as well as the total CN of the gene and the gene paralog, the most likely combination of the CN of the gene base and the CN of the gene paralog base can be determined. If the CN of the gene base is determined as 0, this indicates that the subject has no copy of the wildtype gene haplotype (the haplotype that carries the gene A base at a variant site of interest), and is homozygous for the gene variant haplotype.

The method 600 ends at block 624.

### Execution Environment

FIG. 7 depicts a general architecture of an example computing device 700 configured to determine or identify one or more gene recombinant variants (e.g., GBA variants, CYP21A2 variants) or gene variant status (e.g., carrier, compound heterozygous, or homozygous). The general architecture of the computing device 700 depicted in FIG. 7 includes an arrangement of computer hardware and software components. The computing device 700 may include many more (or fewer) elements than those shown in FIG. 7. It is not necessary, however, that all of these generally conventional elements be shown in order to provide an enabling disclosure. As illustrated, the computing device 700 includes a processing unit 710, a network interface 720, a computer readable medium drive 730, an input/output device interface 740, a display 750, and an input device 760, all of which may communicate with one another by way of a communication bus. The network interface 720 may provide connectivity to one or more networks or computing systems. The processing unit 710 may thus receive information and instructions from other computing systems or services via a network. The processing unit 710 may also communicate to and from memory 770 and further provide output information for an optional display 750 via the input/output device interface 740. The input/output device interface 740 may also accept input from the optional input device 760, such as a keyboard, mouse, digital pen, microphone, touch screen, gesture recognition system, voice recognition system, gamepad, accelerometer, gyroscope, or other input device.

The memory 770 may contain computer program instructions (grouped as modules or components in some embodiments) that the processing unit 710 executes in order to implement one or more embodiments. The memory 770 generally includes RAM, ROM and/or other persistent, auxiliary or non-transitory computer-readable media. The memory 770 may store an operating system 772 that provides computer program instructions for use by the processing unit 710 in the general administration and operation of the computing device 700. The memory 770 may further include computer program instructions and other information for implementing aspects of the present disclosure.

For example, in one embodiment, the memory 770 includes a gene variant or gene variant status determination module 774 for determining or identifying one or more gene recombinant variants or gene variant status (e.g., carrier, compound heterozygous, or homozygous), such as the method 400 described with reference to FIG. 4, the method 500 described with reference to FIG. 5, or the method 600 described with reference to FIG. 6. In addition, memory 770 may include or communicate with the data store 790 and/or one or more other data stores that stores sequence reads processed, read counts determined, Gaussian mixture models, recombinant variants determined, copy numbers of recombinant variants determined, or gene variant status determined.

### ADDITIONAL CONSIDERATIONS

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1.-89. (canceled)

90. A system for determining GBA status comprising:
non-transitory memory configured to store executable instructions; and
a hardware processor in communication with the non-transitory memory, the hardware processor programmed by the executable instructions to perform:
receiving a first plurality of sequence reads generated from a sample obtained from a subject;
aligning the first plurality of sequence reads to a reference genome sequence to obtain a second plurality of sequence reads aligned to GBA gene or GBAP1 gene in the reference genome sequence;
determining a number of the sequence reads of the second plurality of sequence reads aligned to a unique region between GBA gene and GBAP1 gene in the reference genome sequence;
determining a normalized number of the sequence reads aligned to the unique region between GBA gene and GBAP1 gene in the reference genome sequence;
determining a total copy number of GBA gene and GBAP1 gene using a Gaussian mixture model comprising a plurality of Gaussians each representing a different integer copy number, given the normalized number of the sequence reads aligned to the region between GBA gene and GBAP1 gene;
phasing one or more haplotypes originating from GBA gene or GBAP1 gene in a region of GBA gene, or a corresponding region of GBAP1 gene, comprising a plurality of GBA/GBAP1 differentiating bases using sequence reads of the second plurality of sequence reads aligned to the region, or the corresponding region, comprising the plurality of GBA/GBAP1 differentiating bases;
determining a copy number of each of the one or more haplotypes using the total copy number of GBA gene and GBAP1 gene and a number of sequence reads of the second plurality of sequence reads each comprising one or more of the plurality of GBA/GBAP1 differentiating bases that support the haplotype; and
determining a GBA status of the subject using the one or more haplotypes originating from GBA gene or GBAP1 gene in the region of GBA gene, or the corresponding region of GBAP1 gene, and/or the copy number of each of the one or more haplotypes.

91. The system of claim 1, wherein the unique region between GBA gene and GBAP1 gene in the reference genome sequence comprises a unique region about 10 kilobases in length, and/or wherein the unique region between GBA gene and GBAP1 gene in the reference genome sequence comprises chr1:155220429-155230539 of hg38 or a corresponding region of a reference human genome sequence.

92. The system of claim 1, wherein determining the normalized number of the sequence reads aligned to the unique region between GBA gene and GBAP1 gene in the reference genome sequence comprises: determining the normalized number of the sequence reads aligned to the unique region between GBA gene and GBAP1 gene in the reference genome sequence using (1a) a depth of the sequence reads aligned to the unique region between the GBA gene and GBAP1 gene, (1b) a length of the unique region, (2a) a depth of sequence reads of the first plurality of sequence reads aligned to each of a plurality of regions of the reference genome sequence other than a genetic locus comprising GBA gene and GBAP1 gene, and (2b) a length of each of the plurality of regions of the reference genome other than the genetic locus comprising GBA gene and GBAP1 gene.

93. The system of claim 1, wherein the hardware processor is further programmed by the executable instructions to perform: determining a normalized, corrected number of the sequence reads aligned to the unique region between GBA gene and GBAP1 gene in the reference genome sequence from the normalized number of the sequence reads aligned to the unique region between GBA gene and GBAP1 gene in the reference genome sequence using (1) a GC content of the unique region between the GBA gene and GBAP1 gene and optionally (2) a GC content of each of one or more regions of the reference genome sequence other than a genetic locus comprising GBA gene and GBAP1 gene, wherein determining the total copy number of GBA gene and GBAP1 gene comprises: determining the total copy number of GBA gene and GBAP1 gene using the Gaussian mixture model, given the normalized, corrected number of the sequence reads aligned to the region between GBA gene and GBAP1 gene.

94. The system of claim 1, wherein determining the total copy number of GBA gene and GBAP1 gene comprises: determining a copy number of the region between GBA gene and GBAP1 gene using the Gaussian mixture model, given the normalized number of the sequence reads aligned to the region between GBA gene and GBAP1 gene, and wherein the total copy number of GBA gene and GBAP1 gene is the copy number of the region between GBA gene and GBAP1 gene plus two.

95. The system of claim 1, wherein determining the total copy number of GBA gene and GBAP1 gene comprises: determining the total copy number of GBA gene and GBAP1 gene using a Gaussian mixture model and a predetermined posterior probability threshold, given the normalized number of the sequence reads aligned to the region between GBA gene and GBAP1 gene, optionally wherein the predetermined posterior probability threshold is 0.95.

96. The system of claim 1, wherein phasing the one or more haplotypes originating from GBA gene or GBAP1 gene comprises: analyzing linkage information between GBA/GBAP1 differentiating bases of the plurality of GBA/GBAP1 differentiating bases using sequence reads of the second plurality of sequence reads aligned to the region, or the corresponding region, comprising the plurality of GBA/GBAP1 differentiating bases.

97. The system of claim 1, wherein phasing the one or more haplotypes originating from GBA gene or GBAP1 gene comprises: phasing the one or more haplotypes originating from GBA gene or GBAP1 gene using sequence reads of the second plurality of sequence reads each aligned two or more of the plurality of GBA/GBAP1 differentiating bases.

98. The system of claim 1, wherein a sequence read of the second plurality of sequence reads is aligned to the region of GBA gene, or the corresponding region of GBAP1 gene, comprising the plurality of GBA/GBAP1 differentiating bases with an alignment quality score of zero or more.

99. The system of claim 1,
wherein the region of GBA gene, or the corresponding region of GBAP1 gene, comprising the plurality of GBA/GBAP1 differentiating bases is about 1.1 kilobases in length,
wherein the region of GBA gene, or the corresponding region of GBAP1 gene, comprising the plurality of GBA/GBAP1 differentiating bases comprises exons 9-11 of GBA gene, or GBAP1 gene, respectively, and/or
wherein the region of GBA gene, or the corresponding region of GBAP1 gene, comprising the plurality of GBA/GBAP1 differentiating bases comprises p.L483P, p.D448H, c.1263del, RecNciI, RecTL, and c.1263del+RecTL.

100. The system of claim 1, wherein the plurality of GBA/GBAP1 differentiating bases comprises 10 GBA/GBAP1 differentiating bases.

101. The system of claim 1, wherein the one or more haplotypes comprises a wildtype GBA haplotype, a wildtype GBAP1 haplotype, and/or a GBA/GBAP1 hybrid haplotype, optionally wherein the GBA/GBAP1 hybrid haplotype comprises a GBA variant haplotype or a GBAP1 variant haplotype.

102. The system of claim 1, wherein determining the copy number of each of the one or more haplotypes comprises:
determining a likelihood of one copy of a wildtype GBA haplotype is higher than a likelihood of two copies of the wildtype GBA haplotype given the number of sequence reads of the second plurality of sequence reads each comprising one or more of the plurality of GBA/GBAP1 differentiating bases that support the wildtype GBA haplotype; and
determining the copy number of the wildtype GBA haplotype is one.

103. The system of claim 1, wherein the copy number of a wildtype GBA haplotype is one, and wherein the GBA status of the subject comprises a carrier of a GBA variant haplotype.

104. The system of claim 1, wherein the one or more haplotypes comprises four haplotypes, wherein the total copy number of GBA gene and GBAP1 gene is four, wherein the copy number of each of the four haplotypes is one, and wherein GBA status of the subject comprises a carrier of a GBA variant haplotype.

105. The system of claim 1, wherein the one or more haplotypes comprises two or more GBA variant haplotypes, wherein none of the two or more GBA variant haplotypes comprises a GBA base at each of the plurality of GBA/GBAP1 differentiating bases, and wherein the GBA status of the subject comprises compound heterozygous of GBA variant haplotypes.

106. The system of claim 1, wherein the hardware processor is further programmed by the executable instructions to perform: determining a copy number of a GBA base at each of one or more of the plurality of GBA/GBAP1 differentiating bases is zero using sequence reads of the second plurality of sequence reads each comprising a base at the GBA/GBAP1 differentiating base that is not the GBA base, optionally wherein the base at the GBA/GBAP1 differentiating base that is not the GBA base is a GBAP1 base, and optionally wherein determining the GBA status comprises: determining the subject is homozygous of each of the one or more of the plurality of GBA/GBAP1 differentiating bases.

107. The system of claim 1, wherein the hardware processor is further programmed by the executable instructions to perform: generating a user interface (UI) comprising a UI element representing or comprising the GBA status.

108. A method for determining CYP21A2 status comprising:
under control of a hardware processor:
receiving a first plurality of sequence reads generated from a sample obtained from a subject;
aligning the first plurality of sequence reads to a reference genome sequence to obtain a second plurality of sequence reads aligned to CYP21A2 gene or CYP21A1P pseudogene in the reference genome sequence;
determining a number of the sequence reads of the second plurality of sequence reads aligned to CYP21A2 gene or CYP21A1P pseudogene in the reference genome sequence;
determining a normalized number of the sequence reads aligned to CYP21A2 gene or CYP21A1P pseudogene in the reference genome sequence;
determining a total copy number of CYP21A2 gene and CYP21A1P pseudogene using a Gaussian mixture model comprising a plurality of Gaussians each representing a different integer copy number, given the normalized number of the sequence reads aligned to CYP21A2 gene or CYP21A1P pseudogene;
phasing one or more haplotypes originating from CYP21A2 gene or CYP21A1P pseudogene in a region of CYP21A2 gene, or a corresponding region of CYP21A1P pseudogene, comprising a plurality of CYP21A2/CYP21A1P differentiating bases using sequence reads of the second plurality of sequence reads aligned to the region, or the corresponding region, comprising the plurality of CYP21A2/CYP21A1P differentiating bases;
determining a copy number of each of the one or more haplotypes using the total copy number of CYP21A2 gene and CYP21A1P pseudogene and a number of sequence reads of the second plurality of sequence reads each comprising one or more of the plurality of CYP21A2/CYP21A1P differentiating bases that support the haplotype; and
determining a CYP21A2 status of the subject using the one or more haplotypes originating from CYP21A2 gene or CYP21A1P pseudogene in the region of CYP21A2 gene, or the corresponding region of CYP21A1P pseudogene, and/or the copy number of each of the one or more haplotypes.

109. A system for determining a gene recombinant variant comprising:
non-transitory memory configured to store executable instructions and a first plurality of sequence reads generated from a sample obtained from a subject; and
a hardware processor in communication with the non-transitory memory, the hardware processor programmed by the executable instructions to perform:
aligning the first plurality of sequence reads to a reference sequence to obtain a second plurality of sequence reads aligned to a gene or a gene paralog, or a region therebetween, in the reference sequence;
determining a total copy number of the gene and the gene paralog using a Gaussian mixture model comprising a plurality of Gaussians each representing a different integer copy number, given a number of the sequence reads aligned to the gene or the gene paralog, or a region therebetween;
phasing one or more haplotypes originating from the gene, comprising a recombinant variant of the gene, or the gene paralog, or a region of the gene or a corresponding region of the gene paralog, comprising a plurality of gene/gene paralog differentiating bases using sequence reads of the second plurality of sequence reads aligned to the region, or the corresponding region, comprising the plurality of gene/gene paralog differentiating bases; and
determining a copy number of each of the one or more haplotypes using the total copy number of the gene and the gene paralog and a number of sequence reads of the second plurality of sequence reads each comprising one or more of the plurality of gene/gene paralog differentiating bases that support the haplotype.
